(19) United States
(12) Patent Application Publication
(10) Pub. No.: US 2012/0053119 A1
(43) Pub. Date: Mar. 1, 2012

(54) THERAPEUTIC METHOD FOR INCREASING PANCREATIC BETA CELL MASS

(76) Inventors: Joel F. Habener, Newton, MA (US); Zhengyu Liu, Lawrenceville, NJ (US); Tatsuya Yano, Tokyo (JP)

(21) Appl. No.: 13/201,100

(22) PCT Filed: Feb. 11, 2010
(86) PCT No.: PCT/US10/23912

§ 371 Date: Nov. 14, 2011

Related U.S. Application Data
(60) Provisional application No. 61/212,575, filed on Apr. 13, 2009.
(60) Provisional application No. 61/151,682, filed on Feb. 11, 2009.

(51) Int. Cl. A61K 38/26; A61P 3/10; A61K 38/22

(52) U.S. Cl. 514/7.2; 514/6.9; 514/7.3

(57) ABSTRACT

The present invention provides various methods for increasing beta cell mass. In certain embodiments, such methods include steps of administering to a subject an effective amount of: (a) SDF1, a polypeptide having amino acid sequence substantially homologous thereto, or a fragment thereof capable of increasing beta cell survival; and (b) GLP-1 Exendin-4, a polypeptide having amino acid sequence substantially homologous to GLP-1 or Exendin-4, or a fragment of GLP-1 or Exendin-4 capable of promoting beta cell proliferation, whereby beta cell mass is increased in the subject.

### CROSS-REFERENCE TO RELATED APPLICATIONS

This application claims the benefit of U.S. Provisional Application No. 61/151,682, filed Feb. 11, 2009, and U.S. Provisional Application No. 61/212,575, filed Apr. 13, 2009, both of which are incorporated herein by reference in their entirety.

### STATEMENT REGARDING FEDERALLY SPONSORED RESEARCH OR DEVELOPMENT

This invention was made with United States government support awarded by the following agency: USPHS DK30834. The United States has certain rights in this invention.

### FIELD OF THE INVENTION

The present invention relates to the treatment of diabetes. In particular, the invention is directed to methods for increasing beta cell mass in diabetic subjects by administration of therapeutic agents that increase beta cell survival and promote beta cell proliferation.

### BACKGROUND OF THE INVENTION

Beta cell dysfunction and the concomitant decrease in insulin production can result in diabetes mellitus. In Type 1 diabetes, the beta cells are completely destroyed by the immune system, resulting in an absence of insulin producing cells. In Type 2 diabetes, the beta cells become progressively less efficient as the target tissues become resistant to the effects of insulin on glucose uptake. Type 2 diabetes is a progressive disease and beta cell function continues to deteriorate despite on-going treatment with any presently available agent. Thus, beta cells are absent in people with Type 1 diabetes and are functionally impaired in people with Type 2 diabetes.

Beta cell dysfunction currently is treated in several different ways. In the treatment of Type 1 diabetes or the late stages of Type 2 diabetes, insulin replacement therapy is used. Insulin therapy, although life-saving, does not restore normoglycemia, even when continuous infusions or multiple injections are used in complex regimes. For example, postprandial levels of glucose continue to be excessively high in individuals on insulin replacement therapy. Thus, insulin therapy must be delivered by multiple daily injections or continuous infusion and the effects must be carefully monitored to avoid hyperglycemia, hypoglycemia, metabolic acidosis, and ketosis.

Replacement of beta cells can be achieved with pancreatic transplants. Such transplants, however, require finding an appropriate donor, surgical or other invasive procedures for implanting the harvested tissue, and graft acceptance. After transplantation in a person with Type 1 diabetes, on-going immunosuppression therapy is required because cell surface antigens on the beta cells are recognized and attacked by the same processes that destroyed the beta cells originally. Immunosuppressive drugs, such as cyclosporin A, however, have numerous side-effects, including the increase in potential for infection. Transplantation, therefore, can result in numerous complications.

People with Type 2 diabetes are generally treated with drugs that stimulate insulin production and secretion from the beta cells. A major disadvantage of these drugs, however, is that insulin production and secretion is promoted regardless of the level of blood glucose. Thus, food intake must be balanced against the promotion of insulin production and secretion to avoid hypoglycemia or hyperglycemia.

In recent years several new agents have become available to treat Type 2 diabetes. These include metformin, acarbose and troglitazone. However, the drop in hemoglobin A1c obtained by these newer agents is less than adequate, suggesting that they will not improve the long-term control of diabetes mellitus.

Most recently, glucagon-like peptide-1 (GLP-1), a hormone normally secreted by neuroendocrine cells of the gut in response to food, has been suggested as a new treatment for Type 2 diabetes. It increases insulin release by the beta cells even in subjects with long-standing Type 2 diabetes. GLP-1 treatment has an advantage over insulin therapy because GLP-1 stimulates endogenous insulin secretion, which turns off when blood glucose levels drop. When blood glucose levels are high. GLP-1 promotes euglycemia by increasing insulin release and synthesis, inhibiting glucagon release, and decreasing gastric emptying.

In relation to GLP-1, the molecule Exendin-4 is a peptide produced in the salivary glands of the Gila Monster lizard. The amino acid sequence for Exendin-4 is known in the art. Although it is the product of a uniquely non-mammalian gene and appears to be expressed only in the salivary gland, Exendin-4 shares a 52% amino acid sequence homology with GLP-1 and in mammals interacts with the GLP-1 receptor. In vitro, Exendin-4 has been shown to promote insulin secretion by insulin producing cells and, given in equimolar quantities, is more potent than GLP-1 at causing insulin release from insulin producing cells.

In sum, diabetes results from a deficiency of the beta cells of the endocrine pancreas (islets of Langerhans) to produce insulin in amounts sufficient to maintain nutrient homeostasis. In both type 1 diabetes (T1D), and type 2 diabetes (T2D) the beta-cell mass is reduced and the remaining beta-cells are stressed by the glucotoxic effects of prolonged, sustained hyperglycemia. Because a common feature of diabetes is a reduction in beta cell mass, therapeutic treatments and related therapeutics that control and promote beta cell growth and survival are highly desirable in the field of medical science.

### SUMMARY OF THE INVENTION

It is an object of the present invention to overcome or reduce the above stated problems with the prior art by providing a method for increasing beta cell mass. Such a method includes steps of contacting beta cells with an effective amount of: (a) SDF-1 (SEQ ID NO:1), a polypeptide having amino acid sequence substantially homologous thereto, or a fragment thereof capable of increasing beta cell survival; and (b) GLP-1 (SEQ ID NO:2), Exendin-4 (SEQ ID NO:3), a polypeptide having amino acid sequence substantially homologous to GLP-1 or Exendin-1, or a fragment of GLP-1 or Exendin-1 capable of promoting beta cell proliferation, whereby the mass of the beta cells is increased. In certain embodiments wherein a fragment of GLP-1 capable of promoting beta cell proliferation is used, the fragment has the amino acid sequence of SEQ ID NO:4. Preferably, the fragment additionally comprises an amide moiety at the C-terminus of SEQ ID NO:4. In certain embodiments, the beta cells subjected to the method are human pancreatic beta cells.

Further provided is a method for increasing beta cell mass in a subject. Such a method includes steps of administering to a subject an effective amount of: (a) SDF-1, a polypeptide having amino acid sequence substantially homologous thereto, or a fragment thereof capable of increasing beta cell survival; and (b) GLP-1, Exendin-4, a polypeptide having amino acid sequence substantially homologous to GLP-1 or Exendin-4, or a fragment of GLP-1 or Exendin-4 capable of promoting beta cell proliferation, whereby beta cell mass is increased in the subject. In certain embodiments wherein a fragment of GLP-1 capable of promoting beta cell proliferation is administered to a subject, the fragment has the amino acid sequence of SEQ ID NO:4. Preferably, the fragment additionally comprises an amide moiety at the C-terminus of SEQ ID NO:4. In certain embodiments, the subject is a human with Type 1 or Type 2 diabetes and administration occurs via the subcutaneous route.

In another aspect, the invention provides a method for treating diabetes in a subject which includes steps of: (a) obtaining beta cells from the subject being treated or a donor; (b) contacting the beta cells with an effective amount of: SDF-1, a polypeptide having amino acid sequence substantially homologous thereto, or a fragment thereof capable of increasing beta cell survival; and GLP-1, Exendin-4, a polypeptide having amino acid sequence substantially homologous to GLP-1 or Exendin-4, or a fragment of GLP-1 or Exendin-4 capable of promoting beta cell proliferation; and (c) administering the beta cells that were treated in step (b) to the subject. In certain embodiments wherein a fragment of GLP-1 capable of promoting beta cell proliferation is used, the fragment has the amino acid sequence of SEQ ID NO:4. Preferably, the fragment additionally comprises an amide moiety at the C-terminus of SEQ ID NO:4. In certain embodiments, the subject is a human with Type 1 or Type 2 diabetes. The beta cells treated in step (b) may be optionally allowed to increase in mass before administration to the subject.

The invention further encompasses the use of SDF-1, a polypeptide having amino acid sequence substantially homologous thereto, or a fragment thereof capable of increasing beta cell survival, and GLP-1, Exendin-4, a polypeptide having amino acid sequence substantially homologous to GLP-1 or Exendin-4, or a fragment of GLP-1 or Exendin-4 capable of promoting beta cell proliferation for the manufacture of a medicament for treating diabetes in a subject. In certain embodiments wherein a fragment of GLP-1 is used, the fragment has the amino acid sequence of SEQ ID NO:4. Preferably, the fragment additionally comprises an amide moiety at the C-terminus of SEQ ID NO:4.

Other objects, features and advantages of the present invention will become apparent after review of the specification, claims and drawings.

### BRIEF DESCRIPTION OF THE DRAWINGS

FIG. 1. B>A>B hypothesis, injured beta cells induce the expression of SDF-1 that acts on adjacent alpha cells to stimulate cell proliferation and to induce the production of GLP-1. SDF-1 acts in an autocrine mode to promote beta cell survival and GLP-1 acts as a local paracrine hormone to stimulate beta cell growth.

FIG. 1B. The B>A>B Hypothesis. The B>A>B hypothesis, for Beta Cells>Alpha Cells>Beta Cells in a co-operative autocrine/paracrine communicative network, attempts to explain how beta cells regenerate in response to injuries. 1) When beta cells are injured by cytokines and chemokines produced by autoreactive T Cells or by experimental STZ-induced DNA damage, or by glucotoxicity due to prolonged hyperglycemia, pro-apoptosis signaling programs are activated resulting in the production of factors, e.g., SDF-1, insulin. 2) SDF-1 acts on adjacent alpha cells to elicit activation of the cell division cycle and induce the expression of the prohormone convertase PC1/3. These responses result in alpha cell proliferation and a switch in the production of PGDPs from glucagon to glucagon-like peptide-1 (GLP-1). 3) GLP-1 acts back on beta cells to activate cAMP/PKA, Wnt signaling via Ctnnb1/TCF7L2 and beta-cell regeneration (proliferation). 4) SDF-1 also acts on beta cells (autocrine and paracrine) activating anti-apoptosis cell-survival pathways.

FIG. 2. SDF-1 activates Wnt-signaling in isolated islets from the TopGal Wnt signaling reporter mouse. A. Isolated islets express reporter-driven beta-galactosidase. The dark regions mark the expression of beta-galactosidase, an indicator of the activation of the Wnt signaling reporter LacZ transgene. Specificity of SDF-1 for its receptor CXCR4 is shown by the inhibition of beta-galactosidase expression by AMD3100, a specific antagonist of CXCR4. A demonstration that the SDF-1/CXCR4 axis is coupled to the G-protein, Gi/o is shown by inhibition of beta-galactosidase expression with pertussis toxin (PTX). B. Polymerase chain reaction showing changes in beta-galactosidase mRNA levels in islets shown in panel A.

FIG. 3. SDF-1 activates Wnt signaling in INS-1 cells. Wnt signaling is indicated by the value of TOPflash luciferase activity divided by FOPflash activity. A. Time course of the TOPflash:FOPflash ratio of INS-1 cell treated with 1 nM SDF-1. B. Ratio of TOPflash to FOPflash activity in INS-1 cells treated with increasing doses of SDF-1. C. Increasing doses of AMD3100 antagonizes SDF-1 activation of Wnt signaling. All values are relative to the value of the untreated INS-1 cells. Data are normalized for transfection efficiency by co-transfected beta-galactosidase and represent means±S.D. of three experiments.

FIG. 4. Roles of PI3K, Akt, EGF receptor and GSK3beta in the basal level and SDF-1-induced Wnt signaling in INS-1 cells. A. Role of GSK3beta in Wnt signaling in INS-1 cells. Constitutively active GSK3beta (caGSK3b) inhibits TOPflash activity whereas dominant-negative GSK3beta (dnGSK3b) has no effect. B. Roles of Galphai, PI3K, and Akt, in regulating Wnt signaling in INS-1 cells. The Galphai inhibitor PTX, PI3K inhibitor LY294002, and ERK inhibitor PD98059 do not affect basal level TOPflash activity. In contrast, PTX and LY294002, but not PD98059, inhibits SDF-1 induced TOPflash activity. The Akt inhibitor SH-5 inhibits both basal level and SDF-1-induced TOPflash activity. C. Constitutively active Akt (caAkt) stimulates basal endogenous Wnt signaling (TOPflash activity) whereas dominantnegative Akt (dnAkt) inhibits both basal and SDF-1-induced TOPflash activity. All values are relative to the first (leftmost) bar. Statistical significance is depicted as * (p<0.05) when compared with control values (leftmost bar). Data are normalized for transfection efficiency by co-transfected beta-galactosidase and represent means±S.D. of three experiments.

FIG. 5. Inhibition of TCF7L2 or beta-catenin suppresses SDF-1 induced TOPflash activity. A. Dominant-negative TCF7L2 inhibits both basal and SDF-1-induced TOPflash activity. B. SiRNAs to beta-catenin SiRNAs inhibit SDF-1-stimulated Wnt signaling in INS-1 cells. INS-1 Cells were transfected and preincubated with TOPflash and the combination of either siRNAs 1 and 2, or scrambled siRNA and then treated for 4 hrs with 1 nM SDF-1 or control vehicle. Data are normalized for transfection efficiency by co-transfected beta-galactosidase and represent means±S.D. of three experiments. C. SDF-1 stabilizes beta-catenin through GPCR coupled Galphai/o. INS-1 cell cultures were stimulated with SDF-1 for the indicated times. Immunoblot analyses with anti-beta-catenin or anti-unphosphorylated beta-catenin (active beta-catenin). A time-course study shows SDF-1 (1 nM) significantly increased the accumulation.

FIG. 6. Inhibition of apoptosis by SDF-1 in INS-1 and MIN-6 cells is reversed by knock-down of betacatenin. A. Western blot analysis of extracts from MIN6 cells that were mock treated, thapsigargin-treated (1 μM) and/or siRNA transfected (0.25 mg/ml), by using anti-serum against cleaved caspase-3 (upper panel), anti-serum against cleaved PARP (middle panel) and actin antibody (lower panel) as a loading control. B. Caspase-3 activity (fluorescence/mg proteinx105) of INS-1 or MIN6 cells mock treated, thapsigargin-treated (1 μM) and/or siRNA transfected (0.25 mg/ml). Results are presented as mean±SD for n=3 independent experiments, All values are relative to the first (leftmost) bar. Statistical significance is depicted as * (p<0.05) when compared with control values (leftmost bar) and # (<0.05) when compared with 1 mM thapsigargin treatment. C. Representative images are shown for TUNEL staining of INS-1 cells of n=3 independent experiments. Quantification of apoptotic cells is expressed as percent apoptotic cells relative to the total cells counted (lower left panel). D. The proliferation rates of INS-1 cells after SDF-1 or Exd4 treatment are presented as relative BrdU and MTT incorporation values compared to that of control treated cells. Statistical significance is depicted as * (p<0.05) when compared with control values (leftmost bar).

FIG. 7. Schematic model of signaling pathways utilized by SDF-1/CXCR4 in the activation of beta catenin/TCF7L2-mediated transcriptional expression of genes involved in beta cell survival. A direct action of Akt on the stabilization of beta-catenin has been suggested, but remains conjectural.

FIG. 8. Wnt signaling focused array analysis. INS-1 cells were stimulated by SDF-1 (10 nM) for 4 hr. Total RNA was isolated and biotin-labeled complementary RNAs were generated. The Wnt signaling pathway-focused microarray filters (SuperArray Bioscience) were hybridzed with these biotin-labeled targets at 60 degree overnight. The filters were washed and subsequently incubated with alkaline phosphatase-conjugated streptavidin and CDP-substrate. The chemiluminescent images were captured. For quantification, the spot intensity was measured and normalized to the value of the housekeeping gene GADPH. Shown is a representative image of the focused array. The numbers indicate some upregulated target genes (1-10) and some downregulated genes (11-24) as compared to control.

FIG. 9. A. real time RT-PCR results show that SDF-1 4 hr treatment of INS-1 cells enhances beta-catenin mRNA expression by 3-fold and cyclin D1 mRNA expression by 2-fold. Expression values are displayed relative to vehicle-treated control. Data are normalized by using the housekeeping gene GADPH as an internal control and represent means±S.D. of three independent experiments. Statistical significance is depicted as (p<0.01) when compared with control values. B. immunoblot shows endogenous expression of total beta-catenin v.s. tubulin in INS-1 cells 12-48 hr after SDF-1 treatment.

FIG. 10. RIP-SDF-1 transgenic mice (TG) that express SDF-1 in beta cells retain partial glycemic control compared to wild type mice (WT) in response to STZ-induced diabetes. SDF-1 expression results in a 50% improvement in glycemia. Beta cell mass was partially preserved in RIPSDF-1 mice (see FIG. 8).

FIG. 11. Beta cells of neonatal (P3) mice express endogenous SDF-1 (left panels), as well as the SDF-1 receptor, CXCR4 (right panels). SDF-1 expression in beta cells decreases as the islets mature after birth and is undetectable in beta cells by day P30. Insulin=lighter grey, SDF-1 and CXCR4=darker grey.

FIG. 12. Streptozotocin (6 hrs) activates Akt (phospho-Akt) in alpha cells of wild type mice (left panel) and RIP-SDF-1 transgenic mice (middle panel). Akt is also activated in peripheral beta cells, adjacent to alpha cells in transgenic mice. Streptozotocin also stimulates the proliferation of peripheral alpha cells, as stained by Ki67 (right panel).

FIG. 13. Beta cell mass is partially preserved in islets of RIP-SDF-1 mice 2 weeks after STZ-induced diabetes. Preservation/regeneration of beta cells is about 50% of normal mass. Islets of wild-type (WT) mice consist almost entirely of alpha cells. This is alpha cell hyperplasia, not “left-over” alpha cells resistant to STZ.

FIG. 14. Conditioned media obtained from INS-1 beta cells injured by induction of apoptosis in conditions of glucose deprivation (no glucose) or by glucotoxicity (25 mM glucose) for 48 hrs induces the proliferation of alphaTC-1 cells (BrdU incorporation) over a 24 hr incubation.

FIG. 15. Injury of beta cells induces SDF-1 expression. INS-1 or Min6 beta cells were treated for 3 to 20 hrs with the stress/apoptosis inducers, thapsigargin, streptozotocin (STZ), or mixtures of cytokines (listed at the bottom of the right lower panel). SDF-1 mRNA was measured in cell extracts by QPCR. Cytokines also stimulate the secretion of SDF-1 Protein (see new FIG. 21).

FIG. 16. BrdU incorporation cell proliferation assay. Serum starved alpha-TC-1 cells were treated with either PBS (Control) or SDF-1 (10 nM) for 20 hrs, and their proliferation rate was examined by a BrdU incorporation assay. Data represent means±S.D. of three experiments. Statistical significance is depicted as * (p<0.05) when compared with control values (left bar).

FIG. 17. CXCR4, the SDF-1 receptor, is expressed in alpha cells determined by RT-PCR. Shown are comparable expressions of CXCR4 in the clonal cell lines, alphaTC-1, alphaTCdelta(D)PC2 absent PC2 expression, and the beta cell line MIN6 as a positive control.

FIG. 18. Activation of CXCR4 by SDF-1 in alphaTC-1 cells induces phosphorylation of Akt. Shown is a semi-quantitative measurement of the ratio of phospho-Akt to Akt (lower) determined by Western immunoblots (upper).

FIG. 19. SDF-1 induces prohormone convertase PC1/3 expression by one hour after addition of SDF-1 to alphaTC-1 cells. Determined by QPCR. High priority experiments planned are to examine PC1/3 protein expression in isolated mouse islets using Western immunoblot, immunocytochemistry, and bioassays for active PC 1/3.

FIG. 20. SDF-1 induces the production of processed GLP-1 peptide in alphaTC-1 cells. Upper panel: AlphaTC-1 and alphaTCdeltaPC2 cells were incubated for 24 hrs and total GLP-1 secreted was measured in the media. Lower panel: AlphaTC-1 cells were treated with SDF-1 or SDF-1+exendin-4 (Exd4) for the times indicated. Media obtained from the cells was assayed with an RIA specific for the active GLP-1. The findings of increased expression of PC1/3 activity, as evidenced by the production of GLP-1. In the absence of PC2 in alphaTCdPC2 cells is consistent with the findings reported by Wideman et al from the Kieffer lab and by Webb et al in the Steiner lab. It appears that in the absence of PC2, PC13 is expressed. The appearance of GLP-1 between 1 and 4 hrs (lower panel) is consistent with the induction of PC1/3 expression shown above in FIG. 14. A most high priority in the experiments planned is to determine whether SDF-1 (and cytokines) induce the production of GLP-1 peptide in mouse islets, pending the availability of support required to do these studies.

FIG. 21. SDF-1 induces the expression of the GLP-1 receptor (GLP-1R) mRNA in alphaTC-1 cells. Upper panel: alphaTC-1 cells do not express the GLP-1R, whereas, the positive control MIN6 beta cells do express GLP-1R. Lower panel: Addition of SDF-1 (10 nM) to alphaTC-1 cells induces the expression of GLP-1R by 4 hrs. RNA levels were determined by RT-PCR.

FIG. 22. SDF-1 induces the expression of PDX-1 in alphaTC-1 cells. PDX-1 mRNA is induced by 12 hours after the addition of SDF-1 (10 nM), determined by RT-PCR. M=marker lane.

FIG. 23. Model for the activation of PC1/3 expression by SDF-1 in alpha cells. It is envisioned that binding of SDF-1 recruits Janus kinase(s) (Jaks) to CXCR4 resulting in tyrosine phosphorylation of CXCR4, the activation of G-protein, Gi/o, and the phosphorylation of STATs leading to the activation of PI3 kinase (PI3K) and the subsequent activation of the pro-survival kinase Akt. Akt, or other mechanisms activates the nescient helix-loop-helix transcription factor, Nhlh2. STAT(s) and Nhlh2 act together to activate the promoter of the PC1/3 gene. The term activation includes, but not limited to, the possibilities of increased gene expression (mRNA levels), RNA stability, protein phosphorylation, or protein translocation from cytoplasm to nucleus.

FIG. 24. SDF-1 activate Wnt signaling in INS-1 cells. Wnt signaling is indicated by the value of TOPflash luciferase activity divided by FOPflash activity. A. Time course of the TOPflash:FOPflash ratio of INS-1 cell treated with 2 nM SDF-1. B. Ratio of TOPflash to FOPflash activity in INS-1 cells treated with increasing doses of SDF-1. C. Increasing doses of AMD3100 antagonizes SDF-1 activation of Wnt signaling. All values are relative to the first (leftmost) point. Data are normalized for transfection efficiency by cotransfected beta-galactosidase and represent means±S.D. of three experiments. New findings added to this revised application show that SDF-1 activates Wnt signaling in isolated islets from the TopGal Wnt signaling reporter mouse.

FIG. 25. Roles of Galphai, PI3K, Akt, ERK1/2 in regulating Wnt signaling in INS-1 cells. Left panel: The Galphai inhibitor PTX, and the PI3K inhibitor LY294002, but not the ERK1/2 inhibitor PD98059, impair SDF-1-induced, but not basal, TOPflash activity in INS-1 cells. The Akt inhibitor SH-5 inhibits both basal level and SDF-1-induced TOPflash activity. Right panel: Constitutively-active Akt (caAkt) stimulates basal endogenous Wnt signaling (TOPflash activity) whereas dominant-negative Akt (dnAkt) inhibits both basal and SDF-1-induced TOPflash activity. All values are relative to the first (leftmost) bar. Statistical significance is depicted as * (p<0.05) when compared with control values (leftmost bar). All values are relative to the first (leftmost) bar. Data are normalized for transfection efficiency by co-transfected beta-galactosidase and represent means±S.D. of three experiments.

FIG. 26. Cytokines stimulate the production and secretion of SDF-1 in MIN6 clonal beta cells. Min6 cells were treated with cytokines, IL1b, TNFa, IGNg as in FIG. 10, for 17 hrs. SDF-1 protein was measured by ELISA. The n=6 and the * denotes a P value of significance of <0.00073 for cytokine treatment vs. control. Dose-response and time-course studies are in progress. Highest priority in the continuation grant is to repeat these studies in isolated mouse islets.

FIG. 27. SDF-1 activates Wnt-signaling in isolated islets from the TopGal Wnt signaling reporter mouse. A. Isolated islets express reporter-driven beta-galactosidase. The dark regions mark the expression of betagalactosidase, an indicator of the activation of the Wnt signaling reporter LacZ transgene. Specificity of SDF-1 for its receptor CXCR4 is shown by the inhibition of beta-galactosidase expression by AMD3100, a specific antagonist of CXCR4. A demonstration that the SDF-1/CXCR4 axis is coupled to the Gprotein, Gi/o is shown by inhibition of betagalactosidase expression with pertussis toxin (PTX). B. Polymerase chain reaction showing changes in beta-galactosidase mRNA levels in islets shown in panel A.

FIG. 28. Theoretical model of how two distinct GPCR-mediated input signaling pathways, GLP-1/GLP-1R and SDF-1/CXCR4, may converge on downstream beta-catenin/TCF mediated Wnt-signaling at the level of the expression of distinct functional sets of genes, proproliferation and pro-survival. P, phosphorylation state; CoR, Coregulator; R, regulator; RE, response element.

FIG. 29. Cytokines and SDF-1 itself induce SDF-1 expression in human islets ex vivo. 50 human islets were treated with control, cytokine mixture or SDF-1 for 4 hr, mRNA was then extracted and QRT-PCR was performed. Cytokines 1: 2 ng/ml IL-1beta, 10 ng/ml TNF-alpha, 10 ng/ml IFN-gamma. Cytokines 2: 10 ng/ml IL-1beta, 50 ng/ml TNF-alpha, 50 ng/ml IFN-gamma.

FIG. 30. Cytokines induce SDF-1 expression in mouse islets ex vivo. 50 islets per group were treated with vehicle or cytokine cocktail for 4 hr. mRNA were then collected from islets and quantitative RT-PCR was performed to measure SDF-1 mRNA levels. SDF-1 mRNA is up 3-fold after cytokine treatment.

FIG. 31. SDF-1 and GLP-1 agonist, exendin-4 (Exd4), additively prevent loss of INS-1 beta cell mass induced by cytokines. 10 million INS-1 cells were incubated with vehicle or reagents for 6 days and their dry weight was then measured. Data is expressed as relative mass to vehicle treatment. Cytokine mixture (IL1b, TNFa, IFNg), SDF-1 (10 nM) and Exd4 (10 nM).

FIG. 32. SDF-1 and exendin-4 (Exd4) prevent loss of INS-1 beta cell mass induced by the ER stress inducing drug thapsigargin (Thap). 10 million INS-1 cells were incubated with vehicle or reagents for 6 days and their dry weight was then measured. Data is expressed as relative mass to vehicle treatment. Thapsigargin (50 nM), SDF-1 (10 nM) and Exd4 (10 nM).

FIG. 33. SDF-1 and Exd4 additively preserve INS-1 cell numbers in response to serum deprivation. INS-1 cells were incubated with vehicle or reagents and their cell numbers measured and expressed as relative cell numbers as compared to vehicle treatment (vehicle=1.000). SDF-1 (10 nM), Exd4 (10 nM), SDF-1 (10 nM)+Exd4 (10 nM).

FIG. 34. SDF-1 dose-dependently protects INS-1 cells against glucose toxicity, ATP-lite cell viability assay: INS-1 cells were plated in 96 well plates in normal glucose concentration (11 mM) or high glucose concentration (25 mM). For bars 3-8, SDF-1 was added at the indicated concentrations to high glucose concentration well plates at day 0, 2, 4. Cell viability was measured at day 6. N=4.

FIG. 35. SDF-1 receptor antagonist AMD3100 blocks cytoprotective actions of SDF-1 on INS-1 beta cells. ATP lite cell viability assay: INS-1 cells were plated in 96 well plates. SDF-1 (2 nM) and AMD3100 were added at day 0, 2, 4 and cell viability was measured at day 6 using relative light units (RLU) as compared to control. N=6. AMD 3100 partially and dose-dependently blocked SDF-1 mediated cell survival, with an effective concentration of around 1 uM.

FIG. 36. SDF-1 receptor antagonist AMD3100 inhibits SDF-1 induced protection of INS-1 beta cell capacity to secret insulin. Insulin secretion assay: INS-1 cells were plated in 96 well plates. SDF-1 (2 nM) and AMD3100 were added at day 0, 2, 4 and insulin concentration of culture medium was measured at day 6. N=6.

FIG. 37. Cytokines and SDF-1 stimulate GLP-1 production in mouse islets ex vivo. Batches of 50 islets were treated with control, cytokine mixture, or 10 nM SDF-1. Medium was collected in 1 hr and 4 hr. Islet lysate was collected in 4 hr. GLP-1. Immunoreactive protein was measured by radioimmunoassay.

### DETAILED DESCRIPTION OF THE INVENTION

### I. In General

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by any later-filed nonprovisional applications.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. The Invention

As noted above, stromal cell-derived factor-1 (SDF-1) is a chemokine expressed in stromal tissues in multiple organs. Earlier the inventors reported the expression of SDF-1 and its receptor, CXCR4, in the insulin-producing beta cells of the mouse pancreas and determined that the SDF-1/CXCR4 axis is important for beta cell survival through activation of the prosurvival kinase Akt.

Subsequently, activation of downstream Wnt signaling (beta-catenin and TCF7L2) in response to SDF-1 was examined in the islets of Wnt signaling reporter (TopGal) mice and in INS-1 and MIN6 beta cells. Cytoprotection of beta cells by SDF-1 in response to the induction of apoptosis was assessed by caspase 3 and TUNEL assays.

The inventors determined that SDF-1 induces Wnt signaling in beta cells of isolated islets and in INS-1 cells via CXCR4-mediated activation of Galphai/o coupled signaling and the PI3K/Akt signaling cascade resulting in the inhibition of GSK3beta. The key Wnt signaling regulators, beta-catenin and Akt are activated by SDF-1, at the transcriptional and post-translational levels. Specific inhibition of beta-catenin in the Wnt signaling cascade reverses the anti-apoptotic effects of SDF-1. Therefore, SDF-1 appears to promote pancreatic beta-cell survival via activation of Aid and downstream Wnt signaling via the stabilization and activation of beta-catenin/TCF7L2 transcriptional activators. These findings lead to a mechanism for combination SDF-1 and GLP-1-based anti-diabetic therapies by enhancing beta-cell mass through increasing cell survival and proliferation.

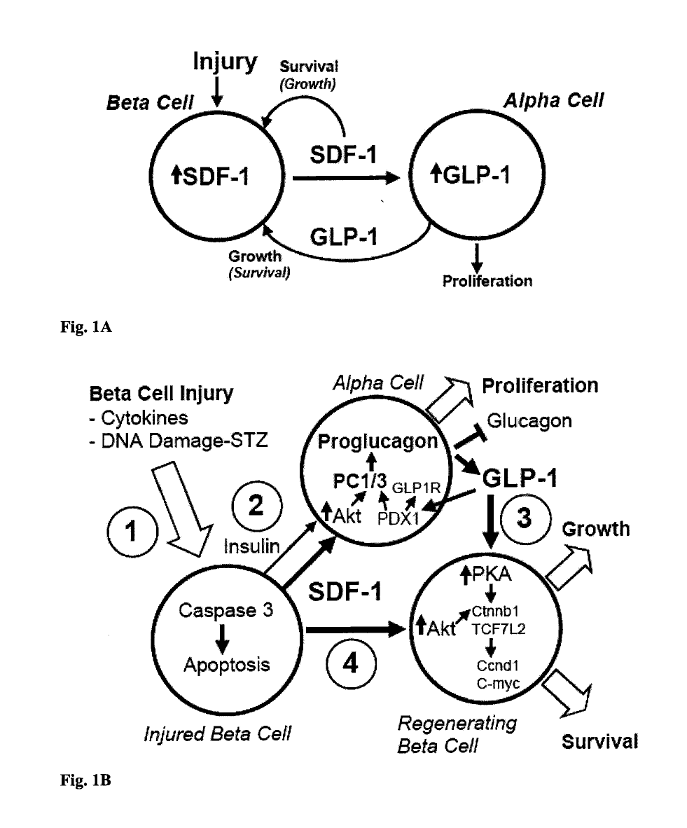

While no one mechanism or mode of action is expressly adopted herein for the presently described and claimed methods, the invention apparently involves intra-islet paracrine interactions amongst the chemokine stromal cell-derived factor-1 (SDF-1) and the glucoincretin hormone glucagon-like peptide-1 (GLP-1) induced by SDF-1 in alpha cells adjacent to beta cells. This hypothesis, termed the B>A>B hypothesis, describes the paracrine signaling from beta to alpha to beta cells in response to beta cell injury (FIG. 1A). Proglucagon encodes both glucagon and glucagon-like peptides (GLPs) and is expressed in the intestinal L-cells and in the pancreatic islet alpha cells. Normally, L-cells process proglucagon into GLPs and the alpha cells produce glucagon. The present invention appears to relate to a novel mechanism in which SDF-1 is induced and secreted by injured beta cells in the islets. SDF-1 acts on adjacent alpha cells to induce the expression of the prohormone convertase, PC1/3, resulting in a switch in the processing of proglucagon from glucagon to GLP-1. As GLP-1 stimulates beta cell proliferation and SDF-1 promotes beta cell survival, the combination of paracrine GLP-1 and autocrine SDF-1 acting synergistically to promote the growth and survival of beta cells.

The glucagon-like-1 peptide hormones are a family of peptides of 31 to 39 amino acids that arise by the post-translational cleavages from proglucagon expressed in the intestine. These hormones were initially recognized as incretin hormones that stimulate glucose-dependent insulin secretion and production by pancreatic beta cells. Proglucagon is also expressed in the brain and the skin, and in the alpha cells of the islets where its cleavage produces the hormone glucagon.

There is a single identified receptor for GLP-1, although circumstantial evidence suggests the possible existence of yet additional receptors(s). The GLP-1 receptor is expressed in a wide variety of tissues including the pancreatic islets, stomach, intestine, brain lung heart, lung, kidney, and skin. The physiologic actions of GLP-1 are numerous. The first described action of GLP-1 is its glucose-dependent insulinotropic actions. GLP-1 stimulates the secretion of insulin and the synthesis of proinsulin in pancreatic beta cells. GLP-1 also exerts pro-proliferative and cytoprotective (anti-apoptotic) actions on beta cells, and may stimulate beta cell neogenesis. Additional actions of GLP-1 include, delayed gastric emptying, suppression of appetite, reduced plasma glucagon levels, and a reduction in hepatic glucose output. Most of the known actions of GLP-1 are antidiabetogenic. The GLP-1 receptor is a G-protein coupled receptor (GPCR) coupled to GalphaS and the cAMP/PKA/CREB signal transduction pathway. Cyclic AMP also activates the guanine exchange factor EPAC and the MEK/ERK1/2 and PI3K/Akt pathways. GLP-1 also indirectly activates the EGF receptor (EGFR) in beta cells by stimulating the local production of betacellulin, an EGF agonist. Signaling via the Ras/Raf/Mek/Erk1/2 and PI3K/Akt pathways stimulates cyclin D1 expression and beta cell replication and inhibits apoptosis.

The recently identified strong genetic association of TCF7L2 with type 2 diabetes may be relevant to GLP-1 signaling in islets. TCF7L2 is a transcription factor that when complexed with betacatenin activates genes downstream of the Wnt signaling pathway, a signaling pathway important in embryonic stem cell amplification, survival, and differentiation. Beta-catenin/TCF7L2 is shown to activate the promoter of the proglucagon gene (Gcg) in intestinal endocrine L-cells). The present inventors recently reported that GLP-1/GLP-1R mediated signaling stimulates the proliferation of beta cells and that such requires the activation of beta-catenin/TCF7L2. Thus, Wnt signaling involving beta-catenin/TCF7L2 may play an active role in both the regulation of proglucagon expression and GLP-1 production in alpha cells and in GLP-1-mediated growth of beta cells.

Stromal cell-derived factor-1 (CXCL12) is a small, secreted 70 amino acid peptide chemokine initially identified in bone marrow-derived stromal cells and now recognized to be expressed in stromal tissues in multiple organs. The SDF-1 receptor, CXCR4, is a GPCR coupled to pertussis toxin sensitive G alphai2 (Gi). The SDF-1/CXCR4 axis is involved in leukocyte trafficking stem cell homing, and in many aspects of development, cell survival, tissue repair and regeneration.

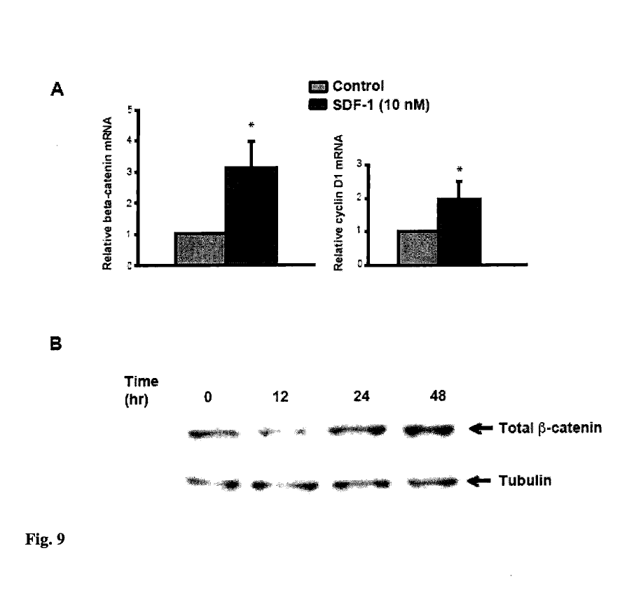

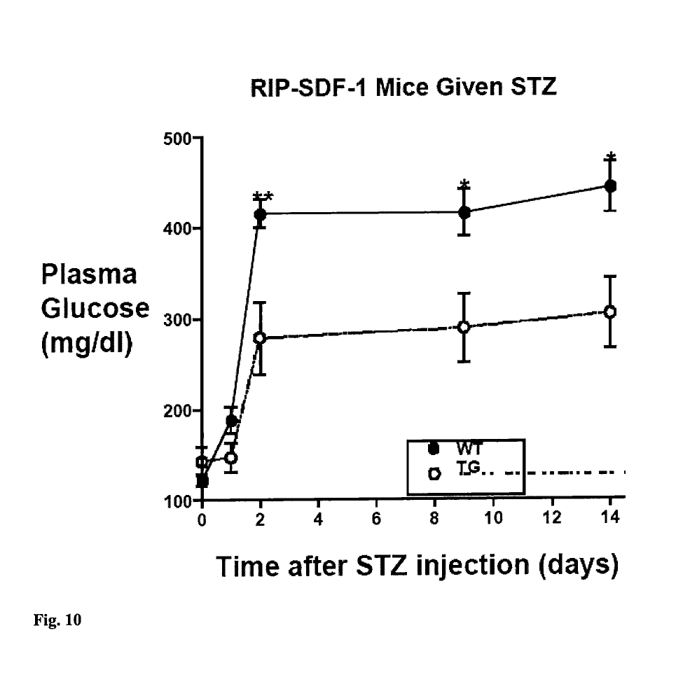

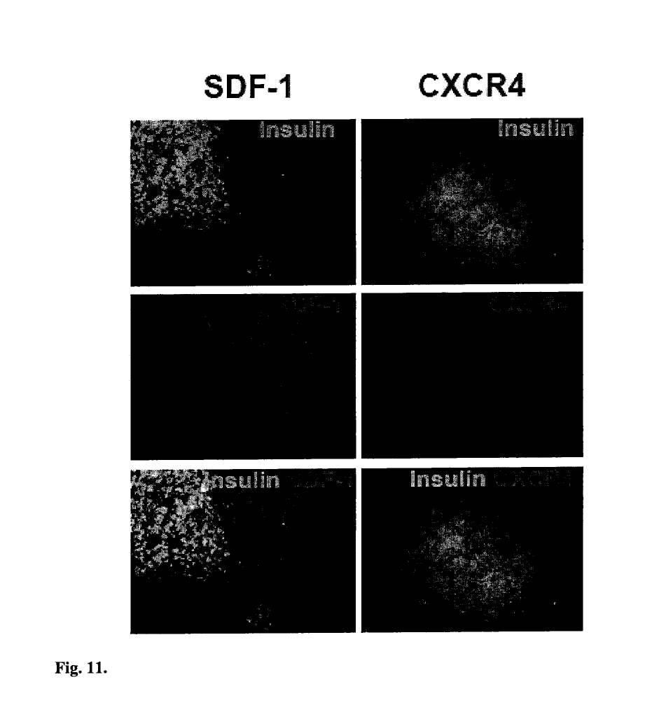

SDF-1 and CXCR4 are expressed in the fetal mouse pancreas, and in the proliferating duct epithelium of the regenerating pancreas of the interferon-gamma mouse. The present inventors reported that transgenic mice expressing SDF-1 in their beta cells (RIP-SDF1 mice) are protected against streptozotocin-induced diabetes, and appear to do so by the activation of the prosurvival protein kinase Akt/PKB and resulting downstream prosurvival, antiapoptotic signaling pathways (see FIG. 10). Notably, in the adult pancreas, beta cells uniformly express the CXCR4 receptor, whereas expression of the SDF-1 ligand is restricted to sparse endothelial and myoepithelial cells within the islets and perivascular and periductal stromal tissues surrounding the islets. In the neonatal pancreas (<10 days) both SDF-1 and CXCR4 are expressed in beta cells suggesting the existence of an autocrine regulation of the SDF-1/CXCR4 axis (FIG. 11). Collectively, these studies strongly support an important role of the SDF-1/CXCR4 axis in pancreas development and regeneration and particularly, in the growth and survival of beta cells. The inventors have recently found that the anti-apoptotic actions of SDF-1 on beta cells requires active Wnt signaling and acts by the stabilization of beta-catenin to enhance gene transcription by TCF7L2.

Accordingly, the present invention is based, at least in part, on the finding that SDF-1 and GLP-1 act additively on beta cells to promote their growth and their survival, and thereby maintain or enhance beta cell mass. Further, both GLP-1 and SDF-1 signaling in beta cells involves downstream Wnt signaling by beta-catenin and TCF7L2. The inventors propose a novel paracrine mechanism in which SDF-1 production from injured beta cells within islets signals to adjacent alpha cells invokes a switch from the production of glucagon to the production of GLP-1. Locally produced GLP-1 acts on injured beta cells to promote their growth. Concurrently, SDF-1 acts back on injured beta cells in an autocrine mode to enhance their survival. Moreover, although both SDF-1 and GLP-1 are likely to be pro-proliferative and anti-apoptotic on beta cells, SDF-1 is predominantly anti-apoptotic and Exendin-4 predominantly pro-proliferative. Both peptides activate the canonical beta-catenin Wnt signaling pathway, but by different upstream mechanisms of the coupling of CXCR4 and GLP-1R to downstream Wnt signaling. Accordingly, the inventors propose that SDF-1/CXCR4 is a strong activator of PI3K and of the prosurvival kinase Akt, resulting in efficient cytoprotection. Akt is an inhibitor of the GSK3/APC/Axin destruction complex that destabilizes beta-catenin, thusly stabilizing beta-catenin/TCF/LEF gene transactivators that regulate survival. GLP-1/GLP-1R may be a strong activator of cAMP/PKA that directly phosphorylates beta-catenin and protects it from destabilization by GSK3/APC/Axin, thereby stabilizing beta-catenin/TCF/LEF gene transactivators that regulate cell proliferation. For example, by this mechanism GLP-1 but not SDF-1, strongly activates the cell division regulators CyclinD1 and c-Myc, and SDF-1 and not GLP-1, strongly activates anti-apoptotic genes such as Bcl2.

As used herein, “subject” means mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

By “diabetes” is meant diabetes mellitus, a metabolic disease characterized by a deficiency or absence of insulin secretion by the pancreas. As used throughout, “diabetes” includes Type 1, Type 2, Type 3, and Type 4 diabetes mellitus unless otherwise specified herein.

As used herein, “administering” or “administration” includes any means for introducing a hormone, chemokine or other chemical agent (collectively “therapeutic agents”) useful in the present methods into the body, preferably into the systemic circulation. Examples include but are not limited to oral, buccal, sublingual, pulmonary, transdermal, transmucosal, as well as subcutaneous, intraperitoneal, intravenous, and intramuscular injection.

A “therapeutically effective amount” means an amount of a hormone, chemokine or other chemical agent that, when administered to a subject for treating a disease, is sufficient to effect such treatment for the disease. The “therapeutically effective amount” will vary depending on the therapeutic agent, the disease state being treated, the severity or the disease treated, the age and relative health of the subject, the route and form of administration, the judgment of the attending medical or veterinary practitioner, and other factors.

For purposes of the present invention, “treating” or “treatment” describes the management and care of a subject for the purpose of combating the disease, condition, or disorder. The terms embrace both preventative, i.e., prophylactic, and palliative treatments. Treating includes the administration of a therapeutic agent of the present invention to prevent the onset of the symptoms or complications, alleviating the symptoms or complications, or eliminating the disease, condition, or disorder.

In the present invention, combinations of therapeutic agents are administered to a subject in a therapeutically effective amount. Such agents can be administered alone or as part of a pharmaceutically acceptable composition. In addition, agents or a composition can be administered all at once, as for example, by a bolus injection, multiple times, such as by a series of injections, or delivered substantially uniformly over a period of time, as for example, using a pump system. Further, the dose of the agent can be varied over time. Agents can be administered using an immediate release formulation, a controlled release formulation, or combinations thereof. The term “controlled release” includes sustained release, delayed release, and combinations thereof.

By “amino acid sequences substantially homologous” to SDF-1, GLP-1 or Exendin-4 is meant polypeptides that include one or more additional amino acids, deletions of amino acids, or substitutions in the amino acid sequence of SDF-1, GLP-1 or Exendin-4 without appreciable loss of functional activity as compared to SDF-1, GLP-1 or Exendin-4 in terms of the ability to effect beta cell survival and proliferation. For example, the deletion can consist of amino acids that are not essential to the presently defined survival and proliferation activities and the substitution(s) can be conservative (i.e., basic, hydrophilic, or hydrophobic amino acids substituted for the same). Thus, it is understood that, where desired, modifications and changes may be made in the amino acid sequence of SDF-1, GLP-1 and Exendin-4, and a protein having like characteristics still obtained. It is thus contemplated that various changes may be made in the amino acid sequence of the SDF-1, GLP-1 or Exendin-4 amino acid sequence (or underlying nucleic acid sequence) without appreciable loss of biological utility or activity and possibly with an increase in such utility or activity.

The term “fragments,” as used herein regarding SDF-1, GLP-1, Exendin-4, or polypeptides having amino acid sequences substantially homologous thereto, means a polypeptide sequence of at least 5 contiguous amino acids of either SDF-1, GLP-1, Exendin-4, or polypeptides having amino acid sequences substantially homologous thereto, wherein the polypeptide sequence has the respective biological functions of SDF-1, GLP-1 and Exendin-4 as described herein. The present fragment may have additional functions that can include antigenicity, binding to GLP-1 receptors, DNA binding (as in transcription factors), and/or RNA binding (as in regulating RNA stability or degradation), targeting to, and/or uptake by subcellular organelles such as mitochondria, nuclei, peroxisomes, endoplasmic reticulum, Golgi, endosomes. Fragments and modified sequences of GLP-1 are known in the art (U.S. Pat. No. 5,614,492; U.S. Pat. No. 5,545,618; European Patent Application, Publication No. EP 0658568 A1; WO 93/25579). Similar fragments and modified sequences of Exendin-4 can be easily extrapolated. It is expected that the following residues in GLP-1 (residue number in superscript) and exendin-4 (residue number in parentheses and superscript) should be included in a fragment since these residues are highly conserved and are important for receptor binding: H⁷⁽¹⁾, G¹⁰⁽⁴⁾, F¹²⁽⁶⁾, T¹³⁽⁷⁾ and D¹⁵⁽⁹⁾. Thus, additional fragments or modified sequences can be easily made that exclude or alter amino acids of GLP-1 and Exendin-4, other than these 5. Because the differentiation activity disclosed herein is easy to assess, the determination that a fragment is within the scope of the invention is routine.

By “contacting” is meant an instance of exposure of the extracellular surface of a cell to a substance at physiologically effective levels. A cell can be contacted by a therapeutic agent, for example, by adding a chemokine to the culture medium (by continuous infusion, by bolus delivery, or by changing the medium to a medium that contains chemokine) or by adding the chemokine to the intracellular fluid in vivo (by local delivery, systemic delivery, intravenous injection, bolus delivery, or continuous infusion). The duration of “contact” with a cell or group of cells is determined by the time the substance is present at physiologically effective levels in the medium or extracellular fluid bathing the cell. In terms of treatment duration, beta cells, as exampled by INS-1 cells, increase in mass in response to the addition of GLP-1 and SDF-1 over seven days of incubation compared to control cells without addition of GLP-1 or SDF-1, in conditions of an environment of injury imposed by addition of thapsigargin or cytokines.

The contacting step in the methods of the present invention can take place in vitro. For example, in a transplantation protocol, ex vivo methods can be employed such that beta cells are removed from a donor (e.g., the subject being treated) and maintained outside the body according to standard protocols well known in the art (see Gromada et al., 1998). While maintained outside the body, the cells could be contacted with the therapeutic agents and the cells subsequently infused (e.g., in an acceptable carrier) or transplanted using methods well known in the art into the donor subject or a subject different from the donor subject.

Alternatively, the contacting step of the present invention can take place in vivo. Methods for administering SDF-1, GLP-1, Exendin-4 or related factors are provided herein. The SDF-1, GLP-1, Exendin-4, or related factors are administered systemically, including, for example, by a pump, by an intravenous line, or by bolus injection (Gutniak et al., 1992; European Patent Application, Publication No. 0619322 A2; U.S. Pat. No. 5,614,492; U.S. Pat. No. 5,545,618). Bolus injection can include subcutaneous, intramuscular, or intraperitoneal routes.

The dosages of SDF-1, GLP-1, Exendin-4, their active fragments or related factors to be used in the in vivo or in vitro methods and processes of the invention preferably range from about 1 pmoles/kg/minute to about 100 nmoles/kg/minute for continuous administration and from about 1 nmoles/kg to about 40 nmoles/kg for bolus injection. More preferably in the in vitro setting, GLP-1 is administered at 1.0-100 ng/ml, Exendin at 0.01-10 ng/ml, and SDF-1 at 1.0-100 ng/ml. In the in vivo setting, once daily injections either intraperitionela or subcutaneous may be given with GLP-1 at 100 nanomole/kgBW, Exendin-4 at 1 nanomole/kgBW and SDF-1 at 10 nanomole/kgBW.

Various exemplary embodiments of compositions and methods according to this invention are now described in the following examples. The following examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### III. Examples

### Example 1

### Materials and Methods

Isolated mouse pancreatic islets Mouse islets were isolated (Lacy PE (1994) Pancreatic islet cell transplant. Mt Sinai J. Med. 61:23-31.) from the pancreata of TopGal reporter mice transgenic for the LEF-LacZ Wnt signaling reporter (DasGupta R, Fuchs E (1999) Development. 1999 126:4557-4568.). Freshly isolated islets were treated for 4 hrs with SDF-1 with and without the addition of the Galphai/o inhibitor pertussis toxin (PTX) or the CXCR4 antagonist AMD3100. Betagalactosidase activity was determined by incubation of the islets with X-gal for 6 hrs. All mouse studies were approved by and in compliance with the MGH IACUC.

Polymerase chain reaction analyses of betagalactosidase mRNA levels in isolated mouse islets Islets from TopGal mice were harvested after treatments with SDF-1, SDF-1+AMD3100, and SDF-1+pertussis toxin. RNA was extracted and beta galactosidase mRNA levels were measured by PCR using a SYBR Green QPCR kit (Stratagene) with the primers as described in Liu Z, Habener J F 2008 Glucagon-like Peptide-1 Activation of TCF7L2-dependent Wnt Signaling Enhances Pancreatic Beta Cell Proliferation. J Biol Chem. 28; 283:8723-8735.

Cell Culture and Transient Transfection INS-1 cells were maintained in RPMI supplemented with 10% heat-inactivated fetal bovine serum, penicillin (100 microgram/ml), and streptomycin (0.25 micrograms/ml), at 37° C. under 5% CO2 and at 95% humidity. MIN6 cells were in DMEM supplemented with 15% fetal bovine serum, penicillin (100 microgram/ml), and streptomycin (0.25 micrograms/ml), at 37° C. under 5% CO2 and at 95% humidity. Transfections were done with lipofectAMINE2000 (Invitrogen).

Wnt Signaling Luciferase Reporter Assay (TOPflash) INS-1 cells were plated into 24-well dishes 24 hr before transfection with TOPflash or the mutant, control reporter, FOPflash (1 microg/well) using LipofectAMINE2000 (Invitrogen). Various concentrations of SDF-1 or AMD3100 were then added to the culture medium 24 h following transfection for the indicated period of time. In studies in which inhibitors were used, LY294002 (50 microM), PD98059 (10 microM) or SH-5 (10 nM) were added concomitantly with SDF-1. In studies in which wild-type (wt), dominant-negative (dn) or contitutively-active (ca) forms of kinase were used, dnTCF7L2, dnGSK3beta, caGSK3beta, dnAkt, or caAkt (0.5 microg/well) was cotransfected with TOPflash. Luciferase activity in transfected cells was determined with a luciferase assay kit (Promega).

SiRNA-mediated knock-down of beta-catenin expression Small interfering RNA fragment (siRNA) against beta-catenin (GenBank accession number NM_{—}053357) were from Dharmacon (siRNA1 cat 0-100628-05, siRNA2 cat #J-100628-06). 50 nM siRNAs were transfected into INS-1 cells using Dharmafect reagent. Transfected cells were grown for 48 or 72 hrs at 37° C. in 5% CO2, then harvested or stained for western blot analysis, caspase-3 assay and TUNEL assay and TOPflash/FOPflash Wnt signaling reporter assays.

Western Immunoblots Membrane immunoblots were prepared from extracts of INS-1 cells and were interrogated with antisera to beta-catenin: total protein (Santa Cruz), and active form with the mutated GSK-3 phosphorylation sites (UpState 8E7).

Thapsigargin-induced apoptosis in INS-1 or MIN6 cells For western immuoblot assay (Cell Signaling), MIN6 cells were seeded into 6 well plates then treated with 1 microM thapsigargin or DMSO control for 16 h and apoptotic activity was measured by Western immunoblotting using antisera specific for cleaved (active) caspase-3 (Cell signaling #9661) and cleaved (active) PARP (Cell signaling #9548). For caspase-3 assay, INS-1 cells or MIN-6 cells in 24-well plates were treated with 1 microM thapsigargin or DMSO for 16 hand caspase-3 activity was determined (Molecular Probes). Caspase-3 activity per well was assessed by a microplate fluorescence reader, and normalized for total protein with the BCA protein assay (Pierce, Roxford, Ill.). In studies examining the anti-apoptotic effects of SDF-1, 10 nM SDF-1 was added concomitantly in the presence or absence of thapsigargin. In studies in which siRNAs were used, beta-catenin. siRNA or scramble siRNA were transfected into cells with Dharmafect reagent one day before thapsigargin treatment.

DAPI (nuclei fluorescence) and TUNEL staining For the terminal deoxynucleotidyl transferase mediated dUTP nick end labeling (TUNEL) assay, cells seeded in 4-well chamber slides (Lab-Tak) were treated with thapsigargin and TUNEL assay performed (DeadEnd™ Fluorimetric TUNEL System kit, Promega).

Cell Proliferation Assay Proliferation of INS-1 cells was determined by incorporation of BrdU into newly synthesized DNA of proliferating cells. Cells in 96-well plates were treated with SDF-1 (10 nM), Exd4 (2 nM) or PBS overnight, then pulse-labeled with BrdU 4 hr. BrdU staining was measured by the DELFIA cell proliferation kit (Perkin Elmer).

MTT Assay Growth of INS-1 cells was determined by the MTT system. Serum starved INS-1 cells in 96-well plates, were treated with SDF-1 (10 nM), Exd4 (2 nM) or PBS for 48 hr and then subjected to MTT assay (Sigma-Aldrich).

Gene expression profiling on focused microarrays INS-1 cells were untreated or treated with 10 nM SDF-1 for 4 hrs. Total RNA was isolated and Biotin-labeled complimentary RNAs were generated using the TrueLabeling-AMP Linear RNA Amplification Kit (SuperArray Bioscience Corp.) The Wnt-signaling pathway-focused microarray filters (118 probes) (SuperArray Bioscience Corp.) were hybridized with these biotin-labeled targets (5 microg/array at 60 C overnight. The filters were washed and subsequently incubated with alkaline phosphatase-conjugated streptavidin and CDPStar substrate. The Chemiluminescent images were captured using Kodak Digital Station 440 (Perkin Elmer). For quantification, the spot intensity was measured and normalized to the value of the housekeeping gene GAPDH. Fold-changes in gene expression levels were determined by comparisons of hybridization densities of SDF-1 treated versus untreated INS-1 cells. Real time RT-PCR was carried out by using the SYBR® Green QPCR kit (Stratagene). Briefly, INS-1 cells were treated with 10 nM SDF-1 or PBS vehicle control for 4 h. Total RNA was reverse-transcribed to cDNA using Super-Script II reverse transcriptase (Invitrogen). Real-time PCR was performed to amplify cyclin D1 and beta-catenin.

Statistical analysis Statistical analysis was done by using an independent samples test. Differences were considered statistically significant at p<0.05.

Amino Acid Sequences Protein sequences of SDF-1, GLP-1 and Exendin-4 are as listed herein below, and can be found in Genbank under their accompanying accession numbers.

SDF-1:
(Accession Number: P48061, positions 24-93;
SEQ ID NO: 1)
VSLSYRCPCRFFESHVARANVKHLKILNTPNCALQIVARLKNNNR
QVCIDPKLKWIQEYLEKALNKRFKM.
GLP-1:
(Accession Number: NP_002045, positions 98-128;
SEQ ID NO: 2)
HAEGTFTSDVSSYLEGQAAKEFIAWLVKGRG.
Also described herein is a 28-36nanopeptide
fragment of GLP-1:
(SEQ ID NO: 4)
FIAWLVKGR,
optionally additionally having an amide moiety at
the C-terminus.
Exendin-4:
(Accession Number: AAB22006; SEQ ID NO: 3)
HGEGTFTSDLSKQMEEEAVRLFIEWLKNGGPSSGAPPPS.
(Optionally with an amide moiety at the
C-terminus.)

### Example 2

### SDF-1/CXCR4 Axis Activates Wnt Signaling in Isolated Mouse Islets

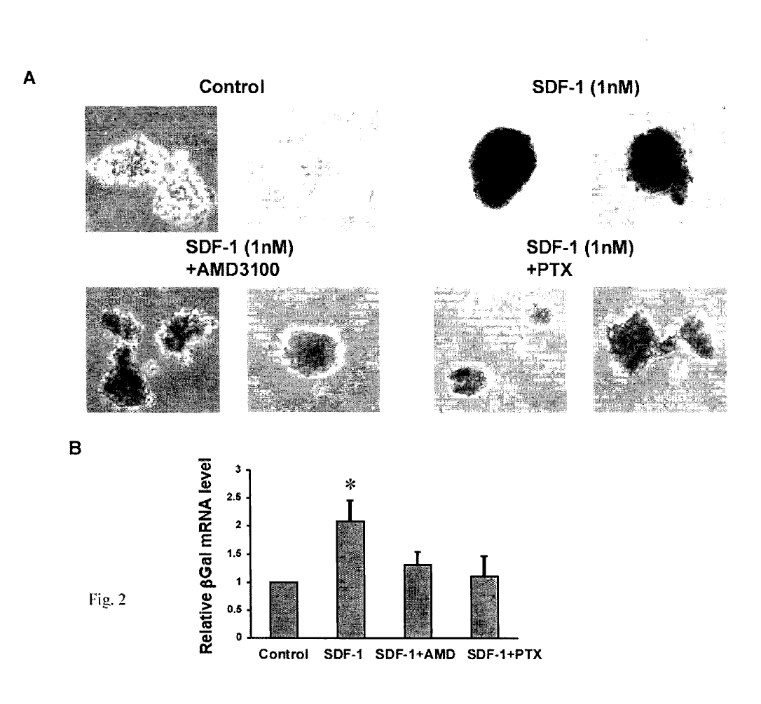

Earlier we reported the expression of SDF-1 in MIN6 beta cells and in the peri-islet stromal tissue and intra-islet endothelial tissue of adult mouse pancreas, as well as expression of the SDF-1 receptor, CXCR4, on both mouse islet beta cells and the MIN6 and INS-1 clonal beta cell lines. Since SDF-1 was recently reported to activate the Wnt signaling pathway in rat neural progenitor cells, we determined whether SDF-1 activates Wnt signaling in pancreatic beta cells. We used isolated islets prepared from the pancreas of the TopGal Wnt signaling reporter mouse in which the expression of beta-galactosidase activity (dark region) indicates activation of the downstream Wnt signaling pathway. Addition of SDF-1 (1 nM) to isolated TopGal islets turned them dark (FIG. 2A). Since the majority of cells (>80%) in islets are beta cells, we conclude that SDF-1 activates Wnt signaling in beta cells. The specificity of the interaction of SDF-1 with its receptor CXCR4 is shown by inhibition of the Wnt signaling response by the specific CXCR4 antagonist, AMD3100, and by pertussis toxin (PTX), an inhibitor of Galphai/o (FIG. 2A). To confirm that SDF-1 activates the transcription of the beta-galactosidase gene, PCR of extracts of the islets was performed to demonstrate corresponding changes in levels of beta-galactosidase mRNA (FIG. 2B).

### Example 3

### SDF-1 Activates Wnt Signaling in INS-1 Cells Via the SDF-1 Receptor

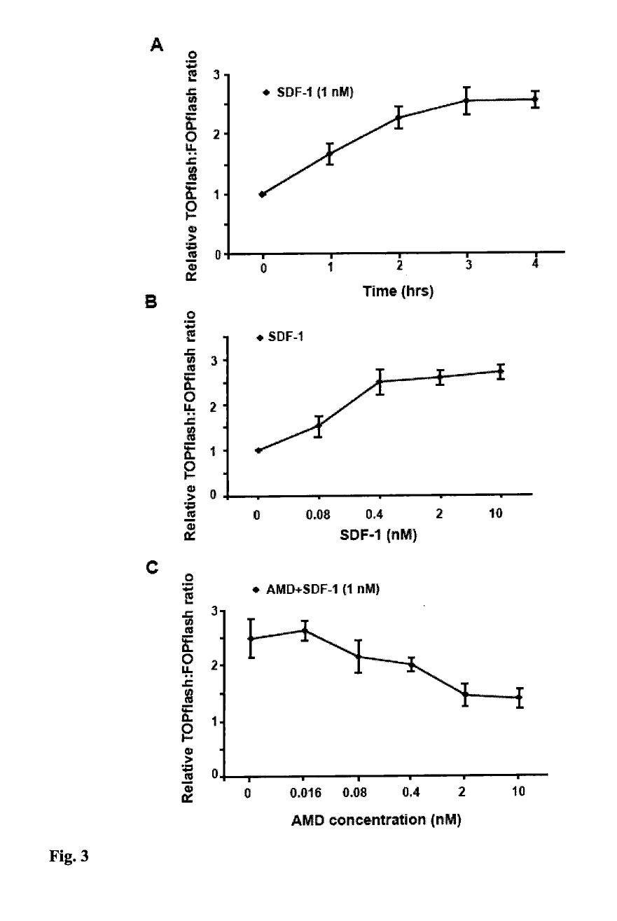

To investigate the mechanisms by which SDF-1/CXCR4 activates Wnt signaling in beta cells we undertook studies in INS-1 cells, a differentiated clonal beta cell line. We examined SDF-1 induction of Wnt signaling using a Wnt signaling reporter assay (TOPflash/FOPflash). The TOPflash and FOPflash constructs contain the luciferase reporter either under the control of consensus TCF7L2-binding sites or mutated TCF7L2-binding sites, respectively. Luciferase activity was measured during 4 hr after addition of 1 nM SDF-1 (FIG. 3A). SDF-1 activated Wnt signaling dosedependently with maximum responses achieved at 0.4 nM (FIG. 3B). SDF-1 activation of luciferase activity was antagonized by coincubation with increasing amounts of the CXCR4 antagonist AMD3100 (FIG. 3C), indicating that the activation of Wnt signaling by SDF-1 occurs via the SDF-1 receptor.

### Example 4

### SDF-1/CXCR4 are coupled to the Galphai/o-PI3K-Akt-GSK3beta signaling pathway

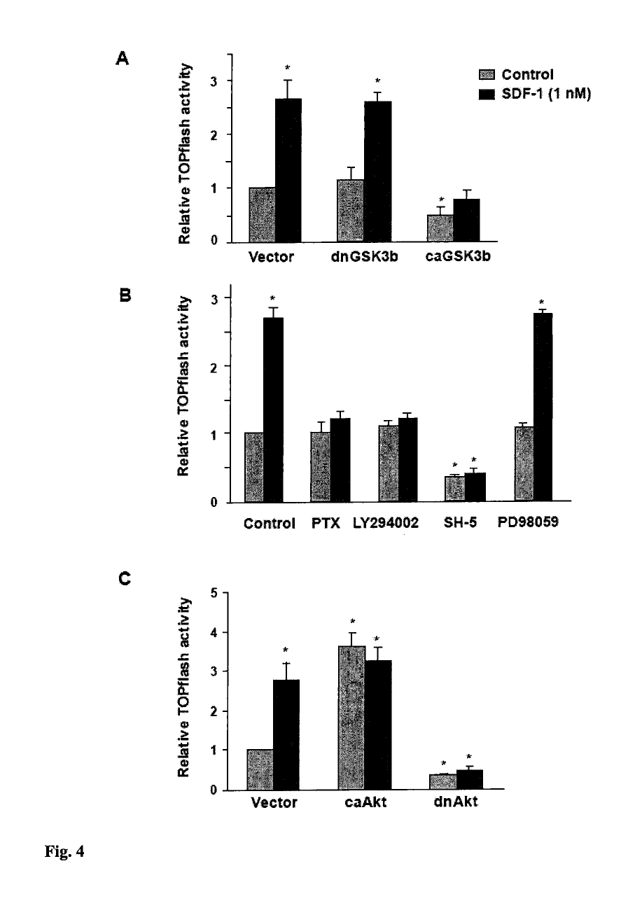

The SDF-1 receptor, CXCR4, is a GPCR coupled to pertussis toxin (PTX) sensitive Galphai2 (Gi). Both PI3K/Akt and MEK/ERK1/2 pathways are activated by SDF-1/CXCR4 in HeLa epithelioid carcinoma cells. SDF-1 promotes pancreatic beta-cell survival in RIP-SDF-1 transgenic mice and in MIN6 and INS-1 clonal beta-cells through the activation of Akt. Because GSK-3beta is inhibited by Akt phosphorylation, which is part of the Wnt signaling pathway, we explored whether the activation of PI3K, Akt, and inactivation of GSK3beta are required for SDF-1-enhanced TOPflash activity. TOPflash reporter assays were performed in INS-1 cells co-transfected with active or inactive form of GSK3beta or pretreated with different kinase inhibitors. Because mutant FOPflash activity was not altered by SDF-1 in repeated experiments we only measured TOPflash activity in the ensuing experiments. Constitutively-active GSK3beta (caGSK3beta) inhibited SDF-1-induced TOPflash activity (FIG. 4A), suggesting inactivation of GSK3beta is required for SDF-1 induced Wnt signaling. Active Galphai/o-PI3K-Akt axis is also required for the effects of SDF-1 on Wnt signaling. SDF-1-stimulated TOPflash activity is inhibited by the Galphai inhibitor pertussis toxin (PTX), PI3K inhibitor LY294002 and the Akt inhibitor SH-5 (FIG. 4B) or dominent negative (dnAkt) (FIG. 4C). A constitutively-active Akt, caAkt, did not augment SDF-1-induced activity indicating that SDF-1 achieved maximum Akt-mediated effects. In contrast, the activation of MAPK was not involved in SDF-1-induced Wnt signaling, since the MAPK inhibitor PD98059 does not affect SDF-1-stimulated TOPflash activity (FIG. 4B).

### Example 5

### Requirements for TCF7L2 and Beta-Catenin for SDF-1-Mediated Wnt Signaling

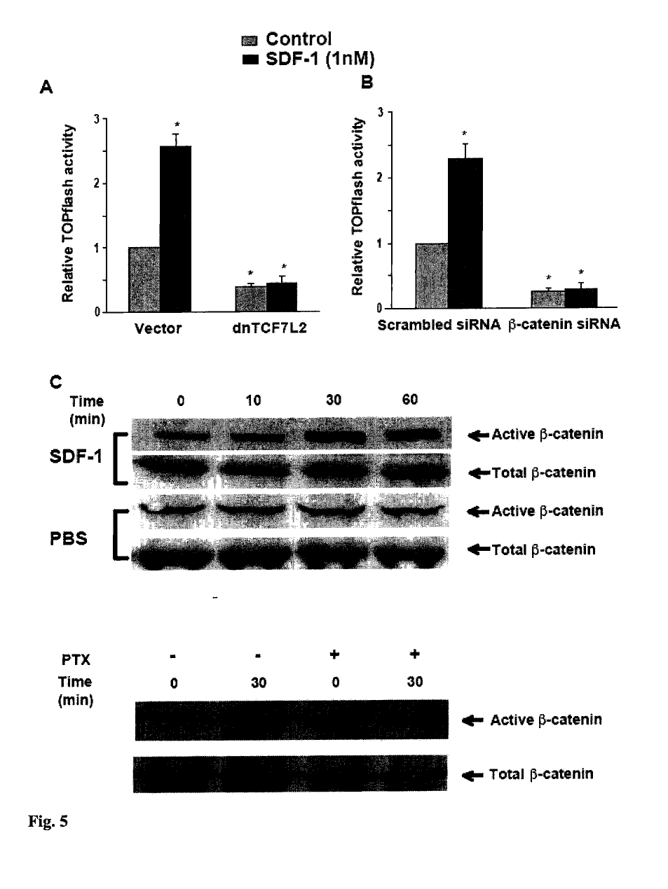

The transcriptional activation of Wnt target genes requires the association of TCF7L2 with nuclear beta-catenin. Therefore, we investigated whether active TCF7L2 is necessary for SDF-1-induced Wnt signaling. INS-1 cells were transfected with a dominant-negative TCF7L2 construct (dnTCF7L2), which lacks the beta-catenin interactive domain and thereby inhibits canonical Wnt signaling. The expression of dnTCF7L2 reduced basal and SDF-1 induced TOPflash activity when compared with cells transfected with control vector (FIG. 5A). In addition to examining the effects of the inhibition of TCF7L2 on SDF-1-mediated Wnt signaling, we examined the requirement for active beta-catenin in this signaling. SiRNAs to beta-catenin markedly inhibited both basal and SDF-1-mediated TopFlash activity (FIG. 5B). To investigate whether SDF-1 increases the stability of beta-catenin, the level of active beta-catenin (unphosphorylated on Ser-33 and Ser-37) was examined. An immunoblot of cells treated with SDF-1 for different period of time was probed with an antibody specific for unphosphorylated beta-catenin. In response to SDF-1, the level of active beta-catenin increased 30 min after the treatment of the INS-1 cells with SDF-1, whereas PBS treatment has no effect. (FIG. 5C). Although no changes were observed in cellular levels of total beta-catenin for up to 60 minutes of SDF-1 treatment of the INS-1 cells, a substantial increase in total beta-catenin was observed after 24-48 hours of SDF-1 treatment (FIG. 9B). Pertussis toxin (PTX) abrogated SDF-1-induced accumulation of active beta-catenin indicating that SDF-1-stabilized beta-catenin is mediated by a GPCR coupled to Galphai/o (FIG. 5C).

### Example 6

### SDF-1 Attenuation of Thapsigargin-Induced Apoptosis in INS-1 and MIN6 Beta Cells Mediated by Beta-Catenin

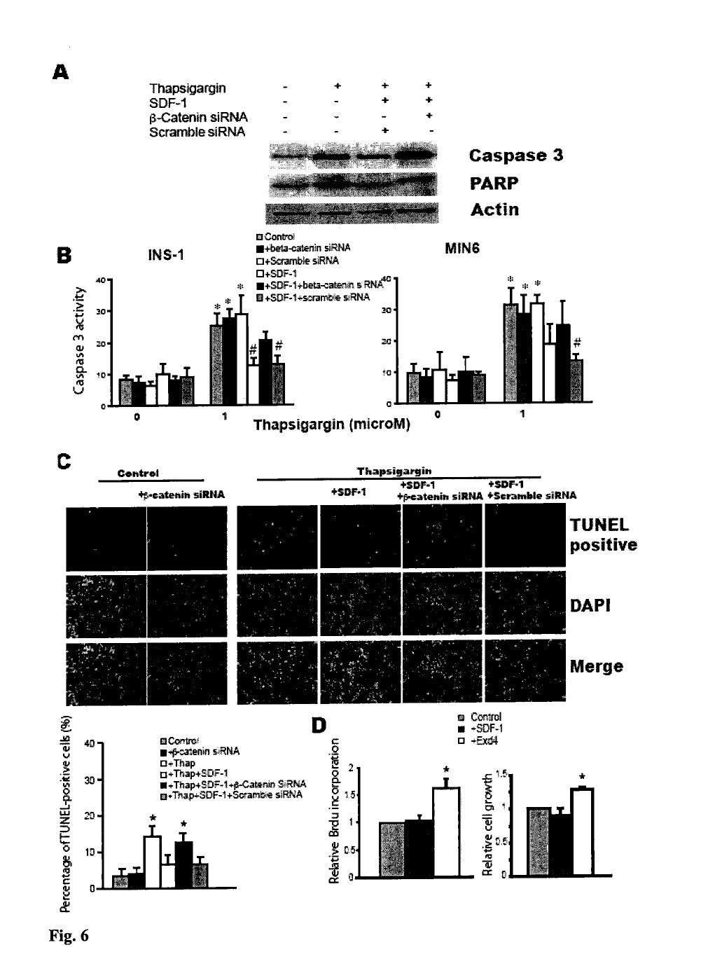

To directly explore the role of downstream Wnt signaling in the cytoprotective actions of SDF-1 on beta cells, the clonal INS-1 and MIN6 beta cells were treated with thapsigargin, a potent inducer of ER stressinduced apoptosis in beta cells. Apoptosis was measured by Western immunoblots that detect specifically the cleaved fragments of caspase-3 and PARP (FIG. 6A). Thapsigargin increased the levels of both cleaved caspase-3 and PARP that was inhibited by SDF-1. Addition of siRNA to beta-catenin, and not scrambled siRNA control, attenuated the inhibition of the cleavages of caspase-3 and PARP (FIG. 6A). The activation of caspase-3 was also measured to evaluate apoptosis during thapsigargin treatment. Thapsigargin induced caspase-3 activity by threefold, and SDF-1 attenuated the proapoptotic effect (FIG. 6B). We also measured caspase-3 activity when endogenous beta-catenin expression was inhibited by siRNA. Reduction of beta-catenin expression with beta-catenin siRNA partially reversed the anti-apoptotic effects of SDF-1 (FIG. 6B). To substantiate these findings, we performed additional apoptosis assay-TUNEL assays (FIG. 6C). Under standard conditions only a few cells were TUNEL positive. The numbers of TUNEL positive cells increased after their treatment with thapsigargin. The effect of thapsigargin was inhibited by co-incubation of the cells with SDF-1. The introduction of beta-catenin siRNA, but not scramble siRNA reversed the cytoprotective actions of SDF-1. The introduction of siRNA alone in INS-1 cells did not induce apoptosis (FIG. 6C and data not shown). These findings indicate that SDF-1 exerts betacatenin-dependent anti-apoptotic effects on INS-1 cells.

In contrast to the GLP-1 agonist, exendin-4 reported earlier [22] SDF-1 exerts no detectable effects on the proliferation of INS-1 cells as determined by the BrdU incorporation and MTT assays (FIG. 6D).

### Example 7

### SDF-1 Regulation of Gene Expression on Focused Microarrays

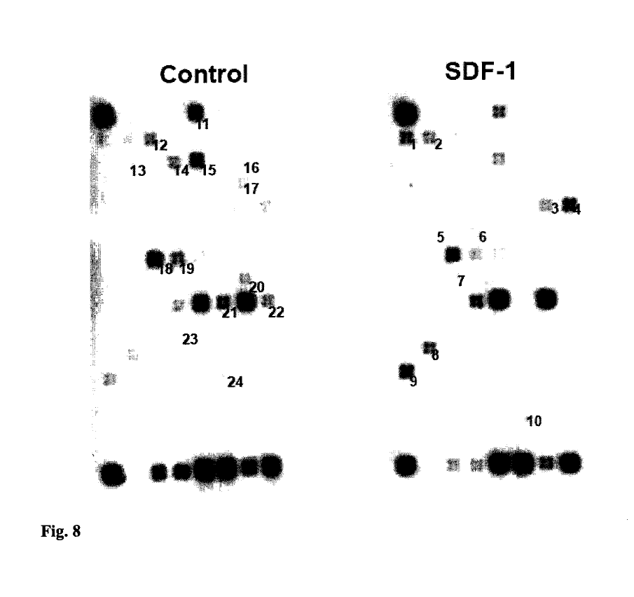

The expression of transcripts in INS-1 cells in response to SDF-1 (10 nM, 4 hrs) was determined on focused arrays of genes expressed in the Wnt signaling pathway. Notably, the expression of beta-catenin (Ctnnb1) mRNA and protein is substantially upregulated (Table 1 and FIG. 8). This finding is consistent with the down-regulation of genes expressing components of the beta-catenin destruction box involved in the degradation of beta-catenin that occurs in the absence of active Wnt signaling; for example, axin, glycogen synthase kinase-3 (Gsk3) and casein kinase 1 (Csnk1). These findings are consistent with the mechanism of Wnt/Fz-induced signaling involving Galphai/o activation of disheveled (Dv1) and the inhibition of the destruction box enzymes GSK3 and Csnk1 that destabilize and degrade betacatenin. Notably, SDF-1 results in the downregulation of cyclin D2 (Ccnd2) and Myc, both involved in cell cycle progression (FIG. 8 and Table 1). Cyclin D1 (Ccnd1) is upregulated by SDF-1 (FIG. 8 and Table 1), confirmed by real time quantitative PCR (FIG. 9A), suggesting that the failure of SDF-1 to stimulate INS-1 cell proliferation (FIG. 6D) is due to a lack of cyclin D2, and not cyclin D1, expression.

TABLE 1
Super array results showed the mRNAs regulated by SDF-1 in INS-1 cells. (See FIG. 8). Quantication of
expression of Wnt target genes regulated by SDF-1 in the superarray was achieved by measuring the ratio of
Intensity of signal of each spot divided by the subtracted intensity by the average of intensity from GAPDH.

| | | | | mRNA fold |
|---|---|---|---|---|
| | Genebank | | | change by |
| Number | number | Symbol | Description | SDF-1 |
| 1 | NM_053357 | Ctnnb1 | Catenin (cadherin associated protein), beta 1 | 1.99 |
| 2 | NM 171992 | Ccnd1 | Cyclin D1 | 1.48 |
| 3 | NM_012953 | Fosl1 | Fos-like antigen 1 | 1.72 |
| 4 | XM_220632 | Foxn1 | Forkhead box N1 | 1.66 |
| 5 | NM_001007597 | Fshb | Follicle stimulating hormone beta | 1.69 |
| 6 | NM_172035 | Fzd2 | Frizzled homolog 2 | 1.43 |
| 7 | XM_215187 | Lrp5_predicted | Low density lipoprotein receptor-related protein 5 (predicted) | 1.72 |
| 8 | NM_021594 | Slc9a3r1 | Solute carrier family 9 (sodium/hydrogen exchanger), | 1.43 |
| | | | isoform 3 regulator 1 | |
| 9 | NM_133524 | Tcfe2a | Transcription factor E2a | 1.61 |
| 10 | NM_001009695 | Wnt7b | Wingless-related MMTV integration site 7B | 1.59 |
| 11 | NM_024405 | Axin1 | Axin1 | −2.58 |
| 12 | NM_022267 | Ccnd2 | Cyclin D2 | −2.29 |
| 13 | NM_053824 | Csnk2a1 | Casein kinase II, Alpha1 | −1.42 |
| 14 | NM_031021 | Csnk2b | Casein kinase 2, beta subunit | −3.03 |
| 15 | NM_019201 | Ctbp1 | C-terminal binding protein 1 | −2.32 |
| 16 | NM-053342 | Cxxc4 | CXXC finger 4 | −1.43 |
| 17 | NM_031820 | Dvl1 | Dishevelled, dsh homolog 1 | −1.92 |
| 18 | NM_017344 | Gsk3a | Glycogen synthase kinase 3 alpha | −1.67 |
| 19 | NM-032080 | Gsk3b | Glycogen synthase kinase 3 beta | −1.51 |
| 20 | NM-012603 | Myc | Myc | −2.55 |
| 21 | NM-017040 | Ppp2cb | Protein phosphatase 2, catalytic subunit, beta isoform | −2.12 |
| 22 | NM_001025418 | Ppp2r1b | Protein phosphatase 2 (formerly 2A), regulatory | −1.71 |
| | | | subunit A (PR 65), beta isoform | |
| 23 | NM_057132 | Rhoa | Ras homolog gene family, member A | −1.32 |
| 24 | NM_053738 | Wif1 | Wnt inhibitory factor 1 | −1.92 |

### DISCUSSION

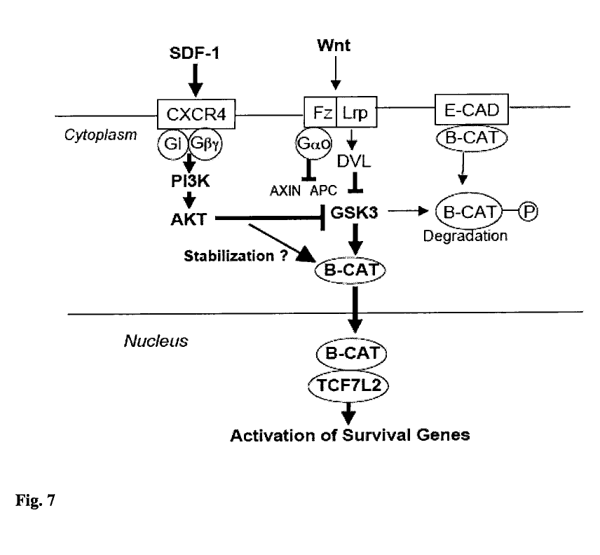

In the above examples 1-8 we tested the hypothesis that SDF-1 activates Wnt signaling in pancreas-derived INS-1 beta cells and isolated islets ex vivo. To our understanding these studies provide novel evidence that SDF-1 enhances Wnt signaling in beta cells and isolated islets. Further, we find that SDF-1 activates Wnt signaling by binding to its receptor CXCR4, a G-proteincoupled receptor that activates Galphai and the downstream PI3K-Akt axis resulting in the subsequent deactivation of GSK3beta and stabilization of beta-catenin (FIG. 7).

A functional role of beta catenin and the transcription factor TCF7L2 in SDF-1 mediated survival and cytoprotection of beta cells was demonstrated by knock-down of beta-catenin with siRNAs. SiRNAs antagonize the SDF-1-mediated inhibition of thapsigargin-induced beta cell apoptosis, indicating that Wnt signaling is obligatory to the prosurvival effects of SDF-1. Modulation of Wnt signaling regulates apoptosis in cancer cell lines and primary cells. Wnt3a and Wnt5a promote proliferation and inhibit apoptosis in HEK293 cells]. Anti-Wnt-1 siRNA inhibits Wnt signaling and induces apoptosis in human breast cancer MCF-7 cells. Increased expression of beta-catenin increases proliferation and inhibits apoptosis of vascular smooth muscle cells (VSMC) following carotid injury in Sprague-Dawley rats.

In a previous report, we demonstrated that SDF-1 promotes islet beta-cell survival using MIN6 and INS-1 clonal beta cells and RIP-SDF-1 transgenic mice and does so through the activation of Akt. Akt and its upstream activating kinase PI3K are well known to modulate Wnt signaling by the phosphorylation and inactivation of GSK3beta. Our findings reported here show that the coupling of SDF-1/CXCR4 signaling to downstream Wnt signaling involves Galphai/o as a major coupling component and that Galphai/o is an activator of PI3K that in turn activates the prosurvival kinase Akt.

It was previously reported that in rat hypothalamus SDF-1 stabilized cytoplasm levels of beta-catenin and induced beta-catenin translocation into the nucleus and binding to the transcription factor TCF7L2 and activated transcription of proproliferative genes such as Ccnd1 (cyclin D1), Ccnd2, Ccnd3, and the transcription factor c-Myc. We found that SDF-1 induces the transcription of beta-catenin in INS-1 cells. In contrast to the well-defined mechanism that accounts for the post-translational regulation of beta-catenin, the transcriptional regulation of betacatenin is less well studied. SDF-1 is among the few ligands that have been shown to activate betacatenin gene transcription. Our discovery raises the interesting possibility of a biphasic upregulation of Wnt and beta-catenin signaling by SDF-1; acutely by posttranslational modifications and longer term by de novo synthesis of betacatenin.

The involvement of Wnt signaling and beta-catenin in pancreas development remains unclear. The expression of a frizzled receptor antagonist in pancreatic progenitor cells of transgenic mice results in a 75% reduction in overall pancreatic mass and a 50% reduction in absolute beta cell numbers. Most recently, human islet-derived precursor cells (hIPCs) were shown to exhibit intense beta-catenin nuclear staining, an indicator of activated Wnt signaling. hIPCs exhibit nuclear beta-catenin throughout exponential cell growth. Beta-catenin signaling is indispensable for hIPC proliferation and during hIPC derivation from islets. Coincidentally, we discovered that inhibition of TCF7L2, an obligatory binding partner of betacatenin, suppressed the proliferation of INS-1 cells and resulting in a reduction in beta cell mass. TCF7L2 appears to be required for maintaining glucose stimulated insulin secretion and beta-cell survival since genetic studies in humans identified a close association of polymorphisms in the TCF7L2 gene and susceptibility towards type 2 diabetes. Our findings indicate that Wnt activation is required for the proproliferative function of GLP-1 agonists and the prosurvival functions of SDF-1 in pancreatic beta cells.

Dissimilarities and Similarities Between SDF-1 and GLP-1-Mediated Wnt Signaling in Beta Cell

These studies of SDF-1 actions, and earlier studies of GLP-1 actions on beta cells demonstrate that both SDF-1 and GLP-1 activate downstream Wnt signaling via betacatenin/TCF7L2 regulated gene transcription. Here we show that downstream Wnt signaling is required for the anti-apoptotic actions of SDF-1 and have reported earlier the requirement of Wnt signaling for the proproliferative actions of GLP-1. Notably, in the studies reported here we find no detectable effects of SDF-1 on beta cell proliferation (FIG. 6D).

There are differences between the interactions of SDF-1/CXCR4 signaling and GLP-1/GLP-1R signaling with the Wnt signaling pathway in beta cells. Although both SDF-1 and GLP-1 activate the downstream pathway of Wnt signaling, consisting of beta-catenin/TCF7L2-mediated gene expression, they do so by way of different pathways of interactions with the more upstream components of the Wnt signaling pathway. SDF-1 inhibits the destruction box of the canonical Wnt signaling pathway consisting of Axin, APC, and the protein kinases, glycogen synthase kinase-3 (Gsk3) and casein kinase-1 (Csnk1). This inhibition of GSK3 and Csnk1 by SDF-1 is likely mediated by the well-known actions of Akt to inhibit these kinases, resulting in the stabilization and accumulation of beta-catenin. In marked contrast to the actions of SDF-1 on beta cells, GLP-1 activates betacatenin/TCF7L2 complexes via the stabilization of beta-catenin by a different mechanism involving the phosphorylation and stabilization of betacatenin by the cAMP-dependent protein kinase A (PKA). PKA activated by GLP-1/GLP-1R phosphorylates beta-catenin on Serine-675 resulting in its stabilization and accumulation. Thusly, unlike SDF-1, GLP-1-induced activation of gene expression by beta-catenin/TCF7L2 in beta cells occurs independently of the destruction box and the activities of GSK3. It also remains possible that beta-catenin may be stabilized by its direct phosphorylation by Akt.

Notably, beta-catenin and TCF7L2 comprise the components of a non-covalent bipartite transcriptional activation complex. Betacatenin is the activation domain and TCF7L2 is the DNA-binding domain of the transactivator. We speculate that different phosphorylations of betacatenin provided by SDF-1 signaling versus GLP-1 signaling result in different conformations of beta-catenin. When different conformers of betacatenin interact with TCF7L2 they confer different conformations to the DNA-binding domains of TCF7L2 resulting in differing affinities of TCF7L2 for its cognate enhancer binding sites on the promoters of various Wnt signaling target genes. Such a combinatorial mechanism could account for the difference in genes regulated by beta-catenin/TCF7L2 in beta cells in response to SDF-1 compared to GLP-1 (FIG. 8).

Wnt-signaling may be a final downstream pathway for both SDF-1 and GLP-1 signaling in beta cells. However, gene expression targets diverge so that SDF-1 predominately regulates genes involved in cell survival, whereas GLP-1 regulates genes involved in cell cycle control (proliferation). If this circumstance proves to be valid, our findings raise the possibility of a dual therapeutic approach for increasing beta cell mass. GLP-1 is predominantly pro-growth and SDF-1 is predominantly pro-survival. Thereby the two peptides may act synergistically to promote both the growth and survival of beta cells, and to conserve, or even enhance, beta cell mass in response to injury.

### Example 8

### Role of Alpha Cells in the Regeneration of Beta Cells

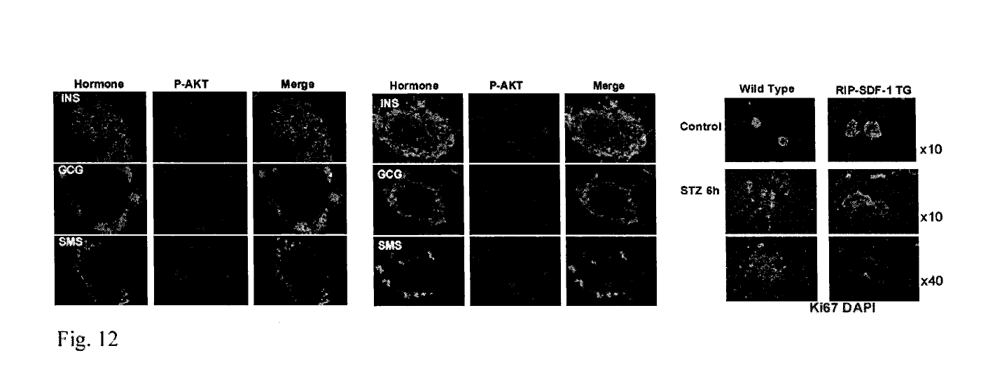

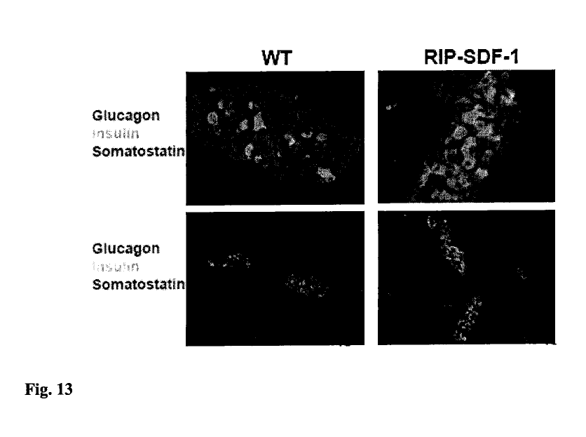

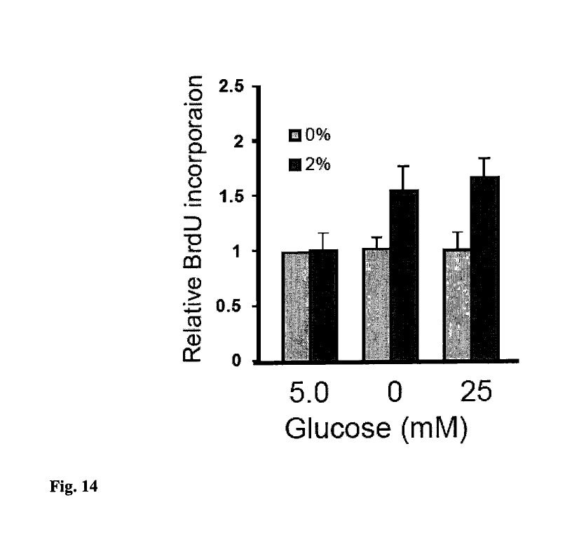

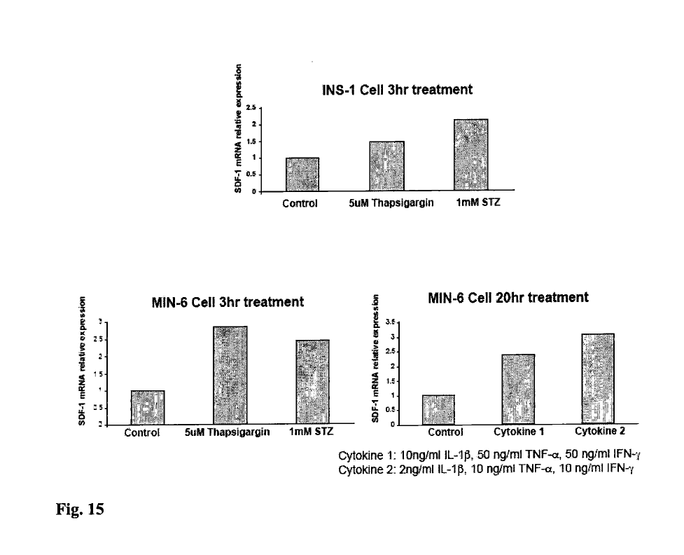

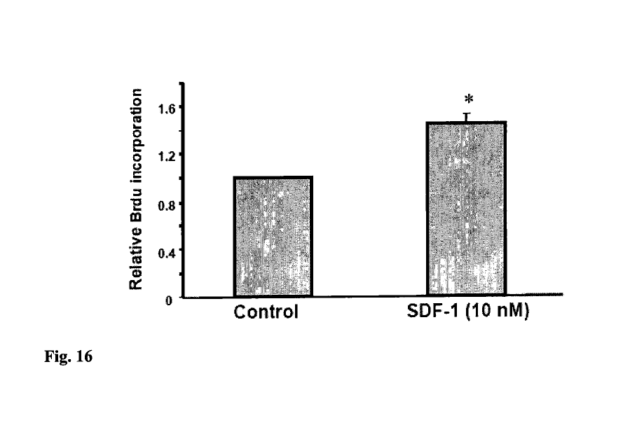

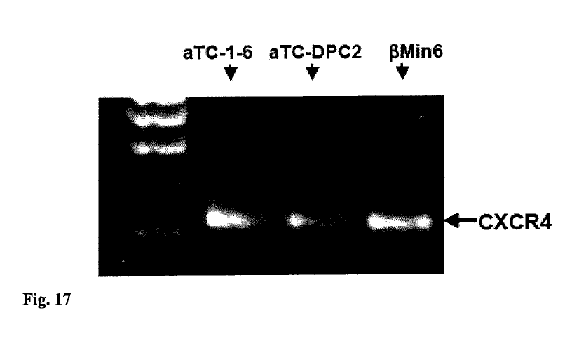

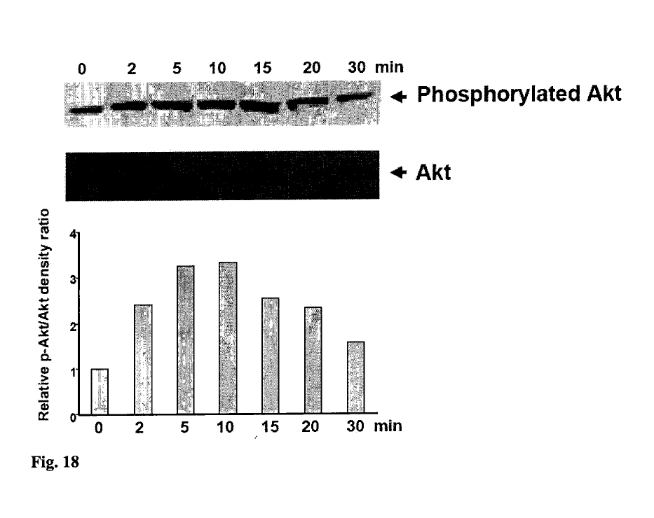

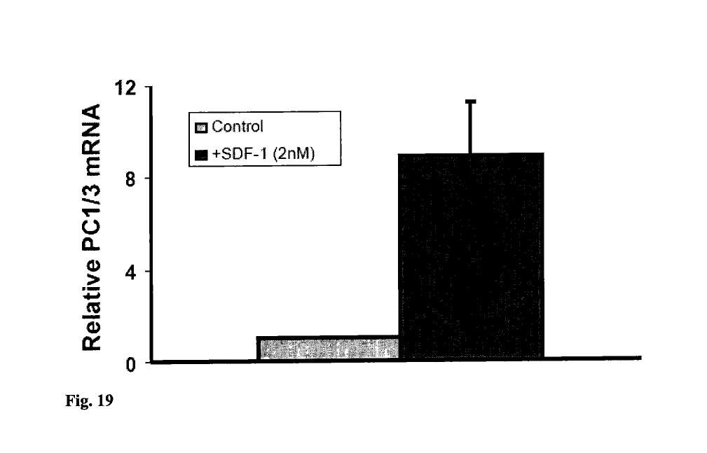

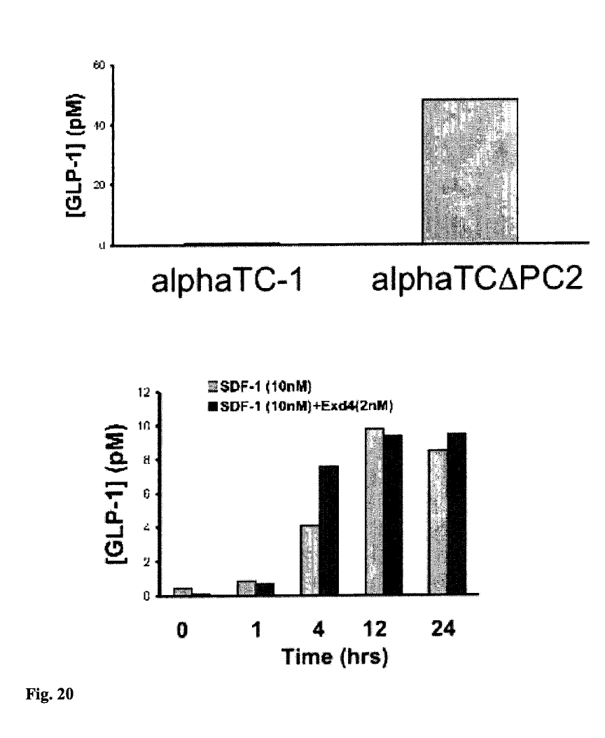

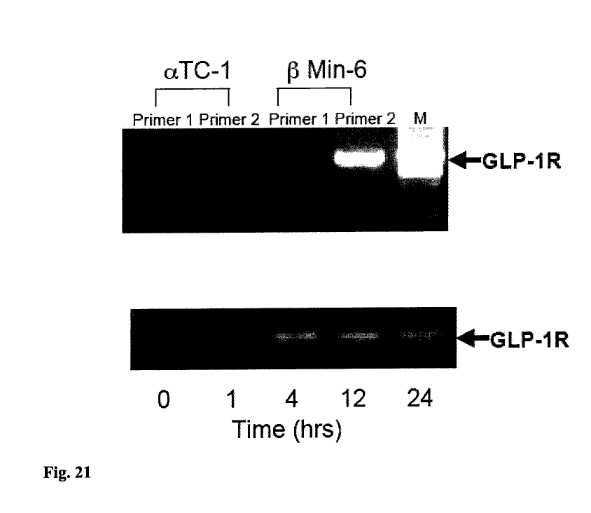

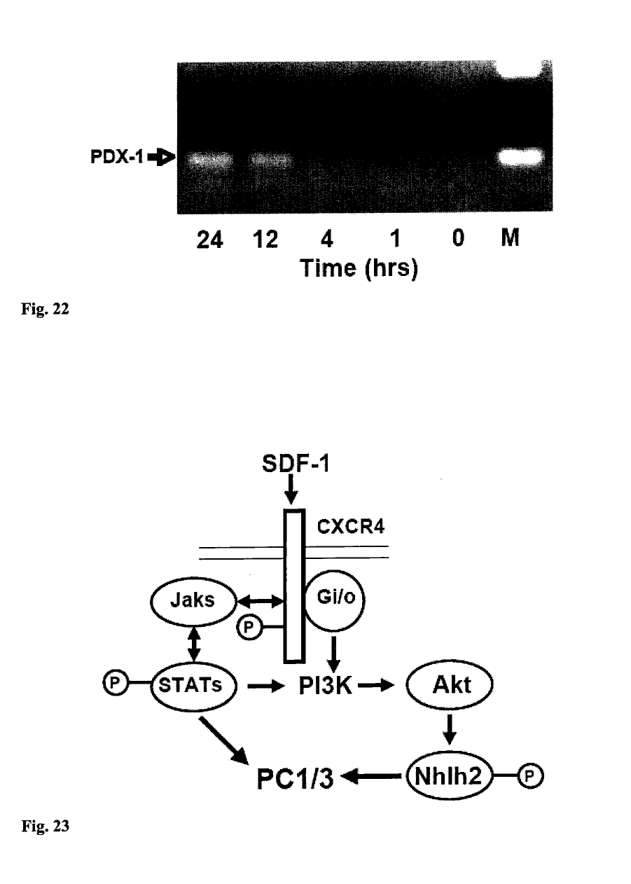

The inventors' studies led to the hypothesis that injured beta cells might be communicating with alpha cells in the islets. The studies were to compare the response of islets to STZ in wild type versus transgenic mice (RIP-SDF-1) over-expressing SDF-1 in beta cells. We observed that 6 hrs after the administration of high dose STZ to normal mice, phospho-Akt appeared in the alpha cells, and not the beta cells of the islets (FIG. 12). Ki67 staining showed proliferation in the alpha cell compartment at the periphery of the islets (FIG. 12, right panel). When the islets were examined 2 weeks after the single dose of streptozotocin the alpha cells had nearly completely replaced the beta cells in the islets (FIG. 13). The presence of continuous SDF-1 production in the RIP-SDF-1 mice maintained much of the beta cell mass, and achieved a 50% improvement in glycemic control (FIG. 10). In preliminary studies we find that conditioned media obtained from INS-1 beta cells in which apoptosis (injury) is induced by either glucose deprivation or by glucotoxicity stimulated the proliferation (BrdU incorporation) of alphaTC-1 cells (FIG. 14). Beta cell injury (INS-1 cells) was induced by glucose deprivation and by glucotoxicity, so as to avoid carryover of injurious agents (e.g., streptozotocin, cytokines) in the conditioned media. Conditioned media, and identical unconditioned control media, were added to alpha TC-1 cells and effects on proliferation were determined by BrdU incorporation. At the same time we found that injured beta cells induce the expression of SDF-1 (FIG. 15). These observations led us to the identification of SDF-1 as a legitimate candidate factor produced by injured beta cells that stimulates the proliferation of alpha cells (FIG. 16). In additional preliminary studies we find that as expected, the SDF-1 receptor, CXCR4, is expressed on alpha cells (FIG. 17) and that SDF-1 activates Akt in alpha cells (FIG. 18). Importantly, SDF-1 induces the expression of PC1/3 (Pcsk1) (FIG. 19) and the production of GLP-1 (FIG. 20) in alpha cells. Surprisingly, SDF-1 activates the expression of mRNAs for the GLP-1R by 4 hrs (FIG. 21) and for PDX-1 by 12 hrs after the addition of SDF-1 to alphaTC-1 cells (FIG. 22). Of note, Herrera has reported from lineage tracing studies that alpha cells arise from precursor cells that once expressed PDX-1. We also find that Nhlh2 is expressed in alphaTC-1 cells (not shown). We postulate that activation of PI3K/Akt by SDF-1 and GLP-1, respectively, result in the induction of PC1/3 in alpha cells (FIG. 23). Thusly, we have preliminary proof-of-principle for the existence of each of the four steps in the paracrine/autocrine B>A>B hypothesis (FIG. 1B).

### Example 9

### Mechanisms of Action of SDF-1 on Beta Cells

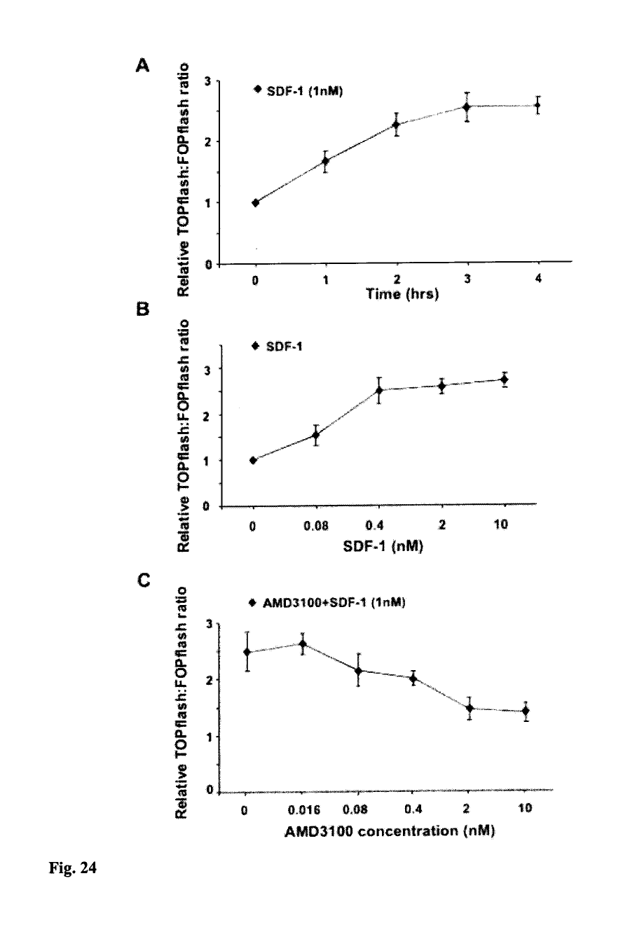

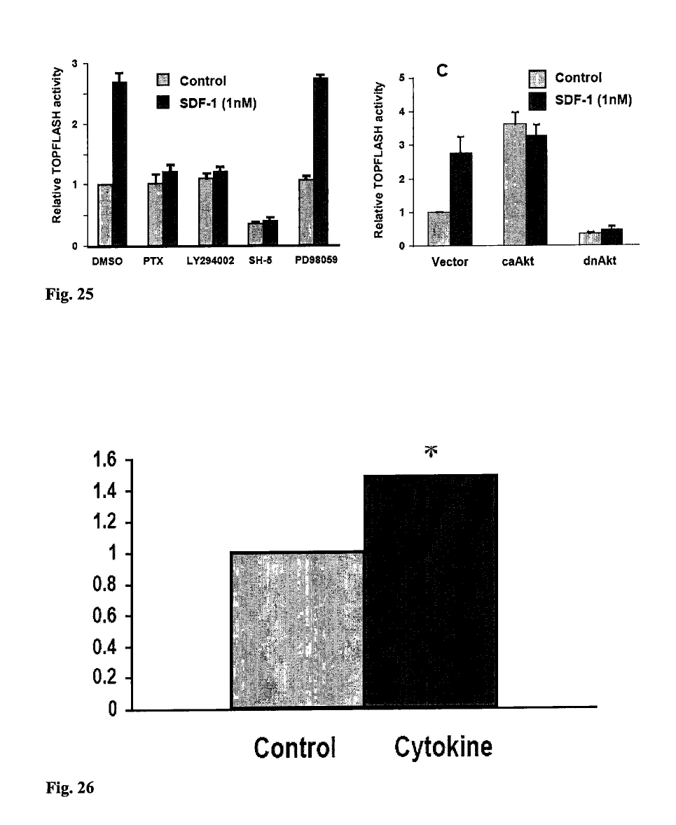

Since we showed that the proproliferative actions of GLP-1 on beta cells requires active beta catenin and TCF7L2 signaling, we wish to examine whether such Wnt signaling is required for the anti-apoptotic actions of SDF-1 on beta cells. Studies of the effects of SDF-1 on Wnt signaling in beta cells are at a preliminary stage of investigation. Using both the Topflash/Fopflash Wnt signaling reporter system in INS-1 cells and islets from the TopGal Wnt signaling reporter mouse, we find that SDF-1 dose- and time-dependently activates the TopFlash reporter (FIG. 25). Moreover, the actions of SDF-1 on Wnt signaling are dependent on the SDF-1 receptor, CXCR4, since reporter activation is inhibited by the CXCR4 antagonist, AMD3100. Further, the activation of the TopFlash reporter requires the activation of the pro-survival kinase, Akt, and likely, the inhibition of GSK3 activity resulting in the stabilization of beta-catenin. (FIG. 26).

### Example 10

### Synergy Between GLP-1 and SDF-1 in Beta Cell Regeneration

Surprisingly, the mitogenic actions of GLP-1 in beta cells requires active downstream Wnt signaling by beta catenin and TCF7L2. SDF-1** agonists enhanced beta cell survival, as determined by attenuation of thapsigargin-induced caspase 3 activity. We tentatively conclude that GLP-1 has strong (Kd 1 nM) pro-proliferative actions and weak (10 nM) cytoprotective actions in INS-1 cells and mouse islets.

### Example 11

### SDF-1 Exerts Antiapoptotic Actions on Beta Cells

In studies of SDF-1 actions on beta cells in vitro and in vivo we observed cytoprotective actions such as inhibition of caspase 3, activation of Bcl2, inhibition of ROS formation in response to apoptosis-inducing agents (thapsigargin, streptozotocin, glucose deprivation, glucotoxicity, cytokines). Moreover, RIP-SDF-1 transgenic mice show enhanced regeneration of beta cell mass after streptozotocin-induced apoptosis, resulting in a 50% attenuation of hyperglycemia (FIG. 10). SDF-1 has no detectable effects on beta cell proliferation, as determined by BrdU incorporation in INS-1 cells, using the identical protocols reported for GLP-1 agonists. These initial findings suggest that although both GLP-1 and SDF-1 act on their specific G-protein-coupled receptors, GLP-1R and CXCR4, on beta cells, GLP-1 is strongly pro-proliferative and SDF-1 strongly pro-survival, and not the reverse. These findings raise the possibility that the combination of GLP-1 and SDF-1 may act synergistically on beta cells to enhance their growth and survival, respectively.

### Example 12

### Mechanisms of Action of SDF-1 on Beta Cells

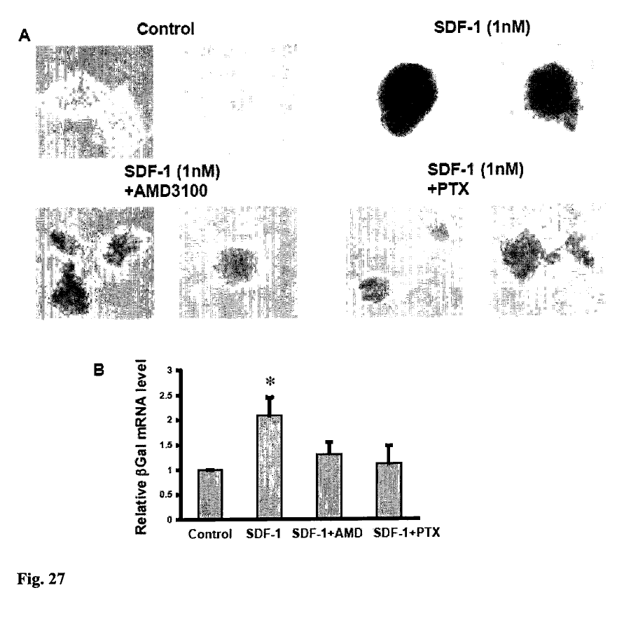

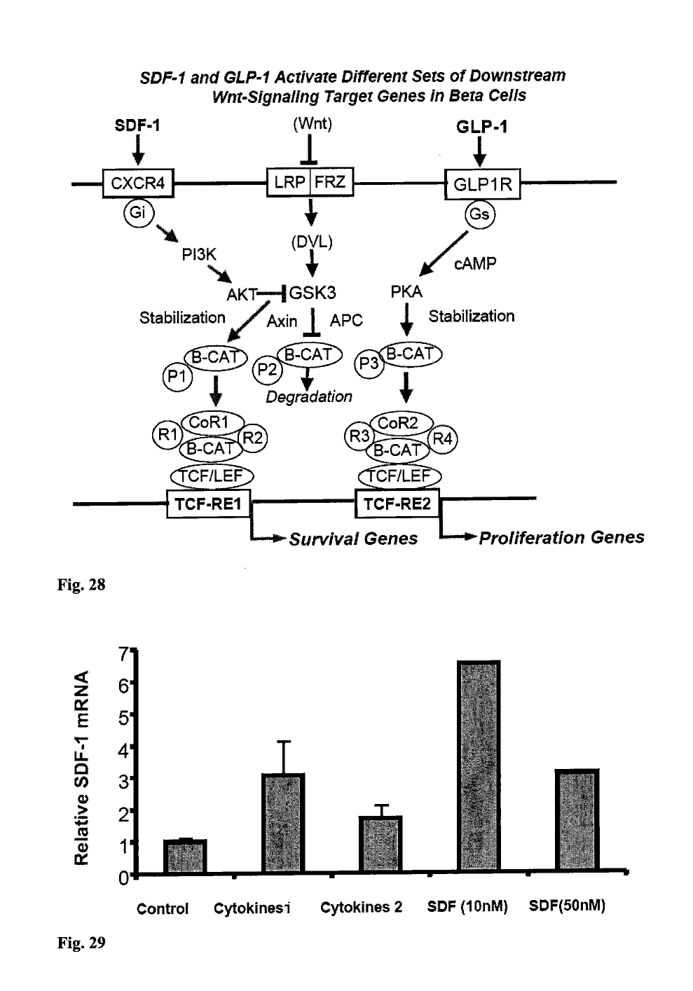

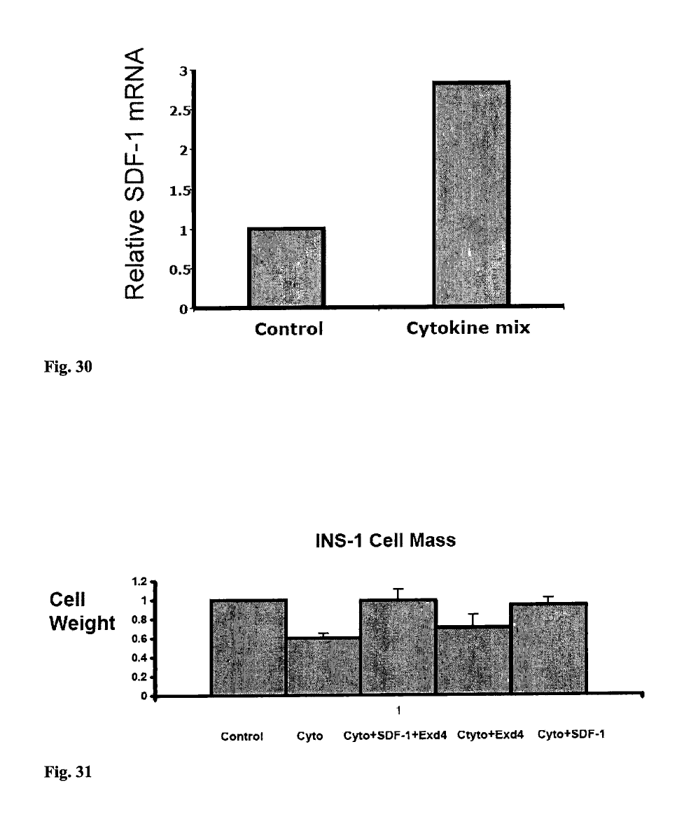

FIG. 28 illustrates a potential model for how SDF-1/CXCR4 (and GLP-1/GLP-1R) signaling couples to beta-catenin/TCF signaling. In INS-1 cells we find that Gi/o is involved in the SDF-1 induction of TopFlash activity because such is inhibited by pertussis toxin (PTX) (FIG. 25). Gi/o is a known strong activator of PI3K. Further PI3K is a known activator of Akt. We have shown in that the anti-apoptotic actions of SDF-1 in INS-1 cells are dependent upon active Akt (FIG. 25). We hypothesize that SDF-1/CXCR4 activates PI3K, which activates Akt, which inhibits GSK3, therefore preventing the phosphorylation of beta-catenin by GSK3 and its consequent ubiquination and degradation. FIG. 27 illustrates that SDF-1 activates Wnt-signaling in isolated islets from the TopGal Wnt signaling reporter mouse. A. Isolated islets express reporter-driven beta-galactosidase. The darkened region marks the expression of betagalactosidase, an indicator of the activation of the Wnt signaling reporter LacZ transgene. Specificity of SDF-1 for its receptor CXCR4 is shown by the inhibition of beta-galactosidase expression by AMD3100, a specific antagonist of CXCR4. A demonstration that the SDF-1/CXCR4 axis is coupled to the Gprotein, Gi/o is shown by inhibition of betagalactosidase expression with pertussis toxin (PTX) B. Polymerase chain reaction showing changes in beta-galactosidase mRNA levels in islets shown in panel A.

### Example 13

### Cytokine-Induced Beta Cell Injury Causes SDF-1 Expression in Isolated Islet Cells

This data we obtained show that injury of beta cells by cytokines induces the expression of SDF-1 in both isolated human and mouse islet cells ex vivo, respectively. Groups of 50 human islet cells were treated with one of vehicle control, two different cytokine mixtures (Cytokines 1: 2 ng/ml IL-1beta, 10 ng/ml TNF-alpha, 10 ng/ml IFN-gamma; Cytokines 2: 10 ng/ml IL-1beta, 50 ng/ml TNF-alpha, 50 ng/ml IFN-gamma), or SDF-1 at one of two different concentrations (10 nM or 50 nM) for 4 hours. SDF-1 mRNA was then extracted and QRT-PCR was performed. The results are shown in FIG. 29. The data show that both treatment with cytokines and treatment with SDF-1 itself induce SDF-1 expression in human islets ex vivo.

We performed a similar experiment using groups of mouse islet cells. Groups comprising 50 islet cells each were treated with either vehicle control or cytokine cocktail for 4 hours. SDF-1 mRNA was then collected from the islets and quantitative RT-PCR was performed to measure SDF-1 mRNA levels. Note that SDF-1 mRNA is up 3-fold after cytokine treatment, demonstrating that cytokine-induced islet cell injury causes increased expression of SDF-1.

### Example 14

### Synergism Between GLP-1 and SDF-1 in Beta Cell Regeneration

The previous examples indicate that GLP-1 and SDF-1 agonists work synergistically to enhance the growth and survival of injured beta cells. Both agonists act through, and require Wnt signaling to recruit the gene products required for the activation of the cell division cycle and to promote anti-apoptosis. This example provides further proof-of-principle that GLP-1 and SDF-1 act synergistically together to preserve and enhance beta cell mass to a greater extent than either agent acting alone.

We have developed an in vitro experimental model of beta cell regeneration using INS-1 cells to test the idea that GLP-1 and SDF-1 agonists act synergistically to conserve, or restore, beta cell mass. INS-1 cells were incubated with various reagent combinations in multi-well plates. Beta cell mass was assessed at the end of the culture by scrape-harvest and weighing of the cell mass, DNA analyses, and MTT colorimetric measure of live cells.

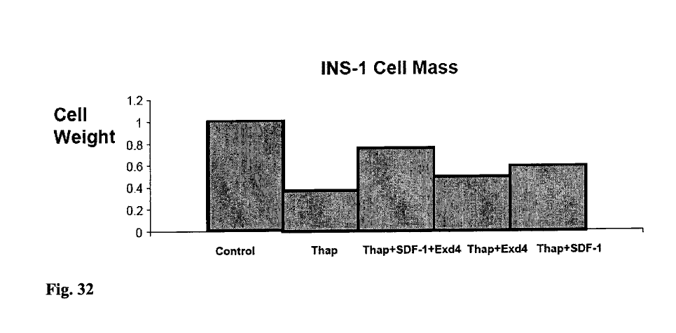

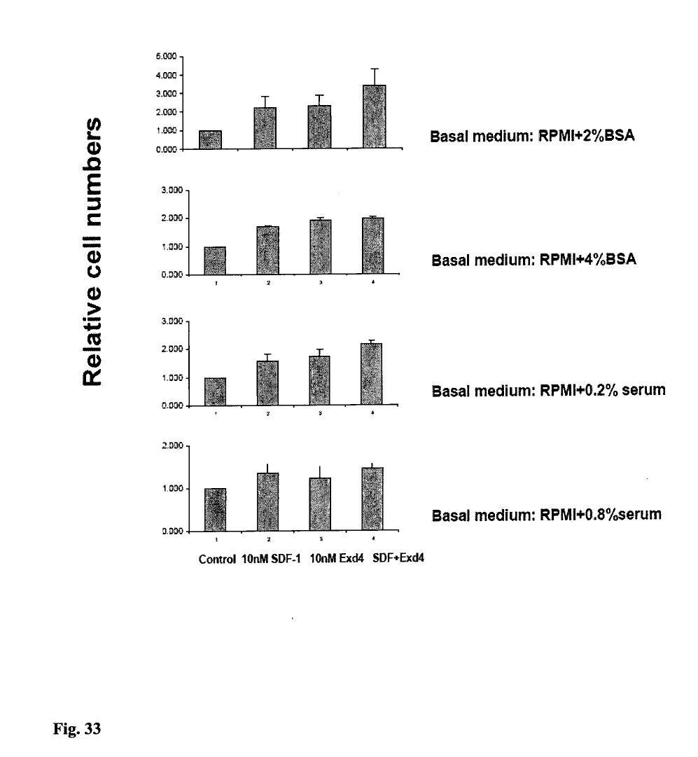

The data show that SDF-1 plus the GLP-1 agonist exendin-4 (Exd4) additively promote conservation of beta cell mass, i.e., protect against INS-1 beta cell loss, in response to such cell loss induced by cytokines (FIG. 31), the ER stress provoking agent thapsigargin (FIG. 32), and the stress of serum deprivation (FIG. 33).

Groups of 10 million INS-1 cells were incubated with vehicle or various reagents for 6 days and their dry weight was then measured to calculate cell weight relative to vehicle treatment control=1.0. Besides a control group, the study included a group of cells exposed to a cytokine mixture (IL1b, TNFa, IFNg); the cytokine mixture plus SDF-1 (10 nM); the cytokine mixture plus Exd4 (10 nM); and the cytokine mixture plus both SDF-1 (10 nM) and Exd4 (10 nM). Treatment with both SDF-1 and Exd4 provided the greatest cell mass recovery in the face of cytokine-induced damage, demonstrating the synergistic effect of using the two agents together (FIG. 31).

Next, we tested the effect of the combination treatment for its efficacy in preventing loss of INS-1 beta cell mass induced by the ER stress inducing drug thapsigargin (Thap). Groups of 10 million INS-1 cells were incubated with vehicle or reagents for 6 days and their dry weight was then measured to calculate cell weight relative to vehicle treatment control=1.0. Besides a control group, the study included a group of cells exposed to thapsigargin (50 nM); thapsigargin plus SDF-1 (10 nM); thapsigargin plus Exd4 (10 nM); and thapsigargin plus both SDF-1 (10 nM) and Exd4 (10 nM). Treatment with both SDF-1 and Exd4 provided substantially greater cell mass recovery in the face of Thap-induced damage than either SDF-1 or EXD4 alone, demonstrating the synergistic effect of using the two agents together (FIG. 32).

Furthermore, we demonstrated that SDF-1 and Exd4 additively preserve INS-1 cell numbers in response to serum deprivation. Four groups of INS-1 cells (control cells treated with vehicle; cells treated with SDF-1 (10 nM), cells treated with Exd4 (10 nM); and cells treated with SDF-1 (10 nM)+Exd4 (10 nM)) were each further subdivided and incubated in four different basal media (RPMI+2% bovine serum albumin (BSA); RPMI+4% bovine serum albumin; RPMI+0.2% serum; and RPMI+0.8% serum). Cell numbers for each of the sixteen different treatment groups were measured and expressed as relative cell numbers as compared to vehicle treatment (vehicle=1.000). The synergistic effect of the combination of SDF-1 and Exd4 was most pronounced under conditions of stress do to serum deprivation (the 0.2% serum and the 2% BSA). Under such conditions, SDF-1+Exd4 again additively ensure the viability of INS-1 cells (see FIG. 33).

### Example 15

### Protective Effect of SDF-1 in INS-1 Cells

We first demonstrate in this example that SDF-1 itself protects against the loss of viability of INS-1 cells induced by glucotoxicity of exposure to high glucose concentrations. Further this experiment shows that these cytoprotective actions of SDF-1 are mediated by the SDF-1 receptor, CXCR4, because the cytoprotective actions are dose-dependently inhibited by the CXCR4-specific antagonist AMD3100.

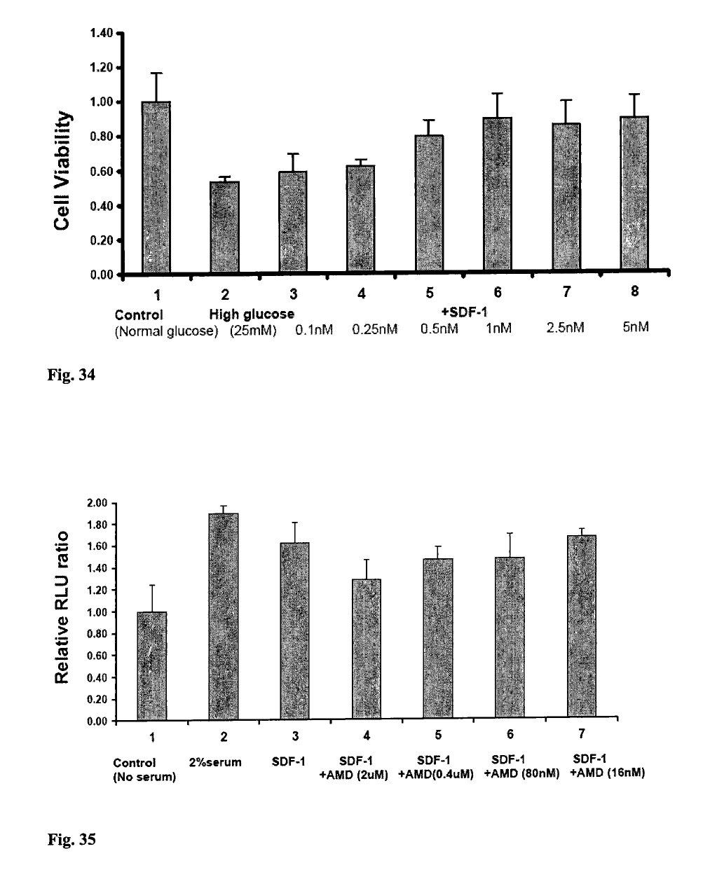

INS-1 cells were plated in 96 well plates in normal glucose concentration (11 mM) or high glucose concentration (25 mM). SDF-1 was added at various concentrations (0.1 nM, 0.25 nM, 5 nM, 1 nM, 2.5 nM, and 5 nM) to high glucose concentration well plates at day 0, 2, 4. Cell viability was measured at day 6, using the ATP-lite cell viability assay (PerkinElmer, Boston, Mass.). The data show that SDF-1 dose-dependently protects INS-1 cells against glucose toxicity (FIG. 34).

We next demonstrate that SDF-1 protects INS-1 cells from dying in response to serum deprivation, and that the cytoprotective actions of SDF-1 are dose-dependently inhibited by the CXCR4-specific antagonist AMD3100.

INS-1 cells were plated in 96 well plates in control medium (no serum) or a medium containing 2% serum. SDF-1 (2 nM) and SDF-1 (2 nM) along with various concentrations of AMD3100 (2 uM, 0.4 uM, 80 nM, 16 nM) were added to different cell groups incubated in serum-free medium at days 0, 2, 4, and cell viability was measured at day 6 using relative light units (RLU) as compared to control. AMD 3100 partially and dose-dependently blocked SDF-1 mediated cell survival, with an effective concentration of around 1 uM (FIG. 35). The results demonstrate that the cytoprotective effects of SDF-1 on cell viability are mediated by the SDF-1 receptor, CXCR4.

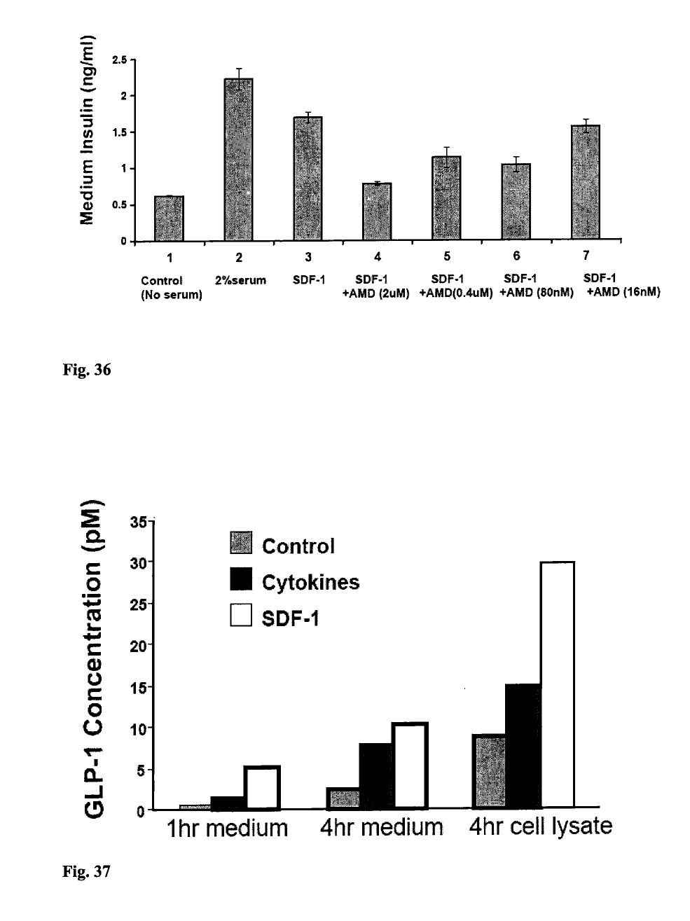

Furthermore, we demonstrate that in INS-1 cells, the SDF-1 receptor antagonist AMD3100 inhibits SDF-1 induced protection of INS-1 beta cell capacity to secret insulin. INS-1 cells were plated in 96 well plates in control medium (no serum) or a medium containing 2% serum. SDF-1 and SDF-1 (2 nM) along with various concentrations of AMD3100 (2 uM, 0.4 uM, 80 nM, 16 nM) were added to different cell groups incubated in serum-free medium at days 0, 2, 4, and the insulin concentration of culture medium was measured by insulin secretion assay at day 6. AMD 3100 partially and dose-dependently blocked SDF-1 mediated insulin production (FIG. 36). The results demonstrate that the cytoprotective effects of SDF-1 on insulin production are also mediated by the SDF-1 receptor, CXCR4.

### Example 16

### Cytokine-Induced Injury of Mouse Islets Induces the Production of GLP-1 by the Islet Cells

In this example, we demonstrate that both injury of islets by cytokines and the exposure of islets to SDF-1 induces the production of GLP-1 by the islets. Batches of 50 islets were treated with a control vehicle, a cytokine mixture, or 10 nM SDF-1. Medium was collected from each batch at 1 hour and 4 hours, and islet lysate was collected in 4 hr. the concentration of GLP-1 contained in each sample collected was measured by radioimmunoassay of the immunoreactive protein. The results are shown in FIG. 37.

Under normal conditions, uninjured islets make glucagon, not GLP-1. The data demonstrate that even in the absence of cytokine-induced injury, SDF-1 can induce the local production of GLP-1 in the islets. We know that the GLP-1 is coming from the alpha cells, because the alpha cells express proglucagon and the beta cells do not. The alpha cells produce the GLP-1 adjacent to the beta cells. Thus, very short range, high concentrations of GLP-1 are produced that can act on injured beta cells to promote their growth, regeneration, and survival.

### Example 17

### Core Nonapeptide Fragment of GLP-1

The inventors have done preliminary studies using GLP-1(28-36), a nine amino acid peptide (the “nonapeptide”) cleaved from the C-terminus of GLP-1 (SEQ ID NO:4). In the studies, the inventors used the peptide having an arginine amide at the C-terminus (FIAWLVKGR-amide). The preliminary data show that the GLP-1(28-36) amide nonapeptide has profound effects on pancreatic beta cell survival and growth. Specifically, infusions of the nonapeptide into mice enhances islet size within the mice. The nonapeptide works both when applied to cells in vitro/ex vivo and when infused into mice in vivo.

Without being limited by any particular theory or mechanism, the cleavage of the nonapeptide from GLP-1 allows targeting of the peptide into mitochondria, where it modulates oxidative phosphorylation. The data indicate that the nonapeptide may be the active peptide core of GLP-1 that acts within mitochodria of liver, fat, pancreatic beta cells, and probably heart and vasculature. The nonapeptide appears to suppress oxidative stress, decrease oxidation, and inhibit apoptosis pathways. Specifically, it appears to prevent glucolipotoxic stress in pancreatic beta cells. Glucolipotoxicity of beta cells is thought to be a major factor in the desensitization of nutrient regulated insulin secretion in type 2 diabetes.

### Example 18

### (Prophetic) Studies of SDF-1 in Streptozotocin-Induced Mouse Models of Diabetes

The potential pro-proliferative and pro-survival actions of GLP-1 and SDF-1 may also be illustrated in mouse in vivo models. We plan to use two mouse strains for models of STZ-induced beta cell injury and diabetes: 1) RIP-SDF1 mice that heterotopically express SDF-1 in beta cells. 2) TopGal mice that report Wnt signaling.

RIP-SDF-1 mouse model. The experimental approach will be to administer Exd4 (1 nanomole/kg, ip. daily, X10d) to STZ-diabetic RIP-SDF1 mice. This will be a reasonable way to demonstrate in vivo the synergism between SDF-1 and GLP-1 (Exd4). We have obtained data on the STZ treatment of RIPSDF1 mice and shown that the expression of SDF-1 in beta cells reduces beta cell apoptosis, enhances phospho-Akt, and partially protects the mice against diabetes (FIG. 10).

By six hours after the administration of STZ there is massive injury to the beta cells and activation of Akt. By two weeks after STZ there is extensive regeneration of beta cells and alpha cell hyperplasia. We feel that the administration of GLP-1 and SDF-1 agonists and antagonists will modify the STZ-induced responses in a meaningful way. We anticipate that Exd4 administered to RIP-SDF-1 mice will further protect beta cells against apoptosis, promote their growth and regeneration, conserve beta cell mass, and further improve glycemia.

Alternatively, the GLP-1R antagonist, exendin(9-39), will be administered (subcutaneous, 10 nmole/kg). In other experiments the SDF-1/CXCR4 antagonist AMD3100 (Mozobil) will be administered to the STZ-treated RIP-SDF1 mice to determine whether it will reverse the resistance of these mice to the development of diabetes. The initial doses of agonists and antagonists will be given at the time of STZ administration. In the event that AMD3100 is ineffective, or causes untoward effects in the mice we will use the monoclonal antibody antagonist 12G5. Both AMD3100 and 12G5 are effective CXCR4 antagonists in mice.

TopGal mouse model. A second series of mouse experiments will employ the TopGal mice rendered diabetic with STZ. These experiments are designed to demonstrate that SDF-1 can be effectively delivered systemically, rather than produced endogenously in beta cells by forced expression in transgenic mice. Exd4 will also be administered to the STZ-diabetic mice. Groups of 5 mice will be given. a) SDF-1b) SDF-1 and AMD3100. c) Exd4). d) SDF-1 and Exd4. e) SDF-1, Exd4, AMD3100, exendin. The design of the experiment is described in (Yano T, Liu Z, Donovan J, Thomas M K, Habener J F: Stromal cell derived factor-1 (SDF-1)/CXCL12 attenuates diabetes in mice and promotes pancreatic beta-cell survival by activation of the prosurvival kinase Akt. Diabetes 56:2946-2957, 2007.). Fasting plasma glucose and insulin levels will be measured at 0, 1, 2, 9, and 14 days. At 2 weeks the mice will be sacrificed and the pancreas removed for measurements of beta cell mass, endocrine hormone immunocytochemistry, and beta galactosidase to index Wnt signaling. For information on signal transduction mechanisms BrdU is given 4 hrs before sacrifice (6 hrs and 72 hrs) to examine beta cell/islet cell proliferation (BrdU and Ki67 staining), apoptosis (TUNEL assay), Akt activation (phospho-Akt staining), and Wnt signaling (beta-galactosidase immunostaining, or Xgal reaction). Details of these procedures are described in (Liu Z, Habener J F. Glucagon-like peptide-1 activation of TCF7L2-dependent Wnt signaling enhances pancreatic beta cell proliferation. J Biol Chem 283, 8723-8735, 2008; Yano T, Liu Z, Donovan J, Thomas M K, Habener J F: Stromal cell derived factor-1 (SDF-1)/CXCL12 attenuates diabetes in mice and promotes pancreatic beta-cell survival by activation of the prosurvival kinase Akt. Diabetes 56:2946-2957, 2007).

If the antagonists Exd(9-39) and AMD3100 fail to antagonize the actions of GLP-1 and SDF-1 agonists in mice in vivo, we propose a conditional knockdown of GLP-1 and SDF-1 actions as an alternative approach. Double transgenic mice will be prepared by crossing RIPCreER mice with mice harboring floxed alleles for the GLP-1R and CXCR4. These mice can then be rendered null for the expression of the receptors for GLP-1 and SDF-1 by the administration of tamoxifen.

### Example 19

### (Prophetic) Additional Studies to Further Establish the Synergism Between GLP-1 and SDF-1 in Beta Cell Regeneration

The previous examples indicate that GLP-1 and SDF-1 agonists work synergistically to enhance the growth and survival of injured beta cells. We propose further studies to demonstrate that GLP-1 is a strong stimulator of the proliferation and SDF-1 is a strong promoter of survival of beta cells, although at sufficiently high concentrations both agents exert both effects.

In vivo studies. Effects of GLP-1, SDF-1, and the two hormones together, on glycemia and beta cell mass in STZ-diabetic mice. An initial mouse model to be studied is the RIP-SDF-1 mouse that expresses SDF-1 in the beta cells. We found that the expression of SDF-1 attenuated the hyperglycemia after STZ to about 50% that of normal control mice (FIG. 10). Mice (8-10 week old, male, C57b1/6) will be rendered diabetic by a single high dose (200 mg/Kg, i.p.) of STZ that results in hyperglycemia (plasma glucose, 400-500 mg/dl, see FIG. 10).

In subsequent experiments we will determine whether the administration of SDF-1 by ip injections has a similar effect in attenuating the glycemia by 50% following STZ, whether exendin-4 alone attenuates glycemia, and whether the combination of SDF-1 and Exd4 achieve euglycemia. Two treatment protocols will be used. Protocol 1 evaluates regeneration after injury of beta cells. Protocol 2 evaluates prevention of injury of beta cells. Protocol 1: Exendin-4 (1.0 umole/kg) and/or SDF-1 (10 umoles/kg) are administered i.p.daily beginning 2 days after STZ administration and confirmation of hyperglycemia. Protocol 2: Exendin-4 and SDF-1 injections are started one day before the treatment with STZ. Plasma glucose, C-peptide, and insulin levels are determined at 0, 1, 2, 9, 14 days after STZ. Groups of six mice are sacrificed 2 days after STZ administration, and the pancreata are removed for analyses of beta cell mass by the usual quantitative morphometric analyses (45,56). Immunostaining is done for insulin, C-peptide, glucagon, somatostatin, PP, PDX-1, Ngn3, and markers of cell proliferation (Ki67, PH3), and apotosis (TUNEL).

If these improvements in glycemia are observed, the experiments will be repeated with the coadministration of specific antagonists of SDF-1 actions (AMD3100 and monoclonal 12G5) (2) and of Exd4 (exendin9-39) to determine whether they block the euglycemic actions of the two hormones. We anticipate that the two hormones together will achieve euglycemic control in otherwise diabetic mice.

Other embodiments and uses of the invention will be apparent to those skilled in the art from consideration from the specification and practice of the invention disclosed herein. All references cited herein for any reason, including all journal citations and U.S./foreign patents and patent applications, are specifically and entirely incorporated herein by reference. It is understood that the invention is not confined to the specific reagents, formulations, reaction conditions, etc., herein illustrated and described, but embraces such modified forms thereof as come within the scope of the following claims.

Sequence Listing. Applicants are submitting as part of this Application a computer readable sequence listing txt file, which is incorporated by reference herein.

What is claimed is:

1. A method for increasing beta cell mass in a subject, comprising administering to the subject an effective amount of (a) SDF-1 (the polypeptide having the amino acid sequence set forth in SEQ ID NO:1), a polypeptide having amino acid sequence substantially homologous thereto, or a fragment thereof capable of increasing beta cell survival; and (b) GLP-1 (the polypeptide having the amino acid sequence set forth in SEQ ID NO:2), Exendin-4 (the polypeptide having the amino acid sequence set forth in SEQ ID NO:3), a polypeptide having amino acid sequence substantially homologous to GLP-1 or Exendin-4, or a fragment of GLP-1 or Exendin-4 capable of promoting beta cell proliferation, whereby beta cell mass is increased in the subject.

2. The method according to claim 1, wherein the fragment of GLP-1 capable of promoting beta cell proliferation administered to the subject has the amino acid sequence set forth in SEQ ID NO:4.

3. The method according to claim 2, wherein the fragment of GLP-1 additionally comprises an amide moiety at the C-terminus of SEQ ID NO:4.

4. The method according to claim 1, wherein said subject is a human with Type 1 diabetes.

5. The method according to claim 1, wherein said subject is a human with Type 2 diabetes.

6. The method according to claim 1, wherein the step of administering to said subject is via subcutaneous injection.

7. A method for increasing beta cell mass, comprising contacting beta cells with an effective amount of (a) SDF-1 (the polypeptide having the amino acid sequence set forth in SEQ ID NO:1), a polypeptide having amino acid sequence substantially homologous thereto, or a fragment thereof capable of increasing beta cell survival; and (b) GLP-1 (the polypeptide having the amino acid sequence set forth in SEQ ID NO:2), Exendin-4 (the polypeptide having the amino acid sequence set forth in SEQ ID NO:3), a polypeptide having amino acid sequence substantially homologous to GLP-1 or Exendin-4, or a fragment of GLP-1 or Exendin-4 capable of promoting beta cell proliferation, whereby the mass of said beta cells is increased.

8. The method according to claim 7, wherein the fragment of GLP-1 capable of promoting beta cell proliferation with which the beta cells are contacted has the amino acid sequence set forth in SEQ ID NO:4.

9. The method according to claim 8, wherein the fragment of GLP-1 additionally comprises an amide moiety at the C-terminus of SEQ ID NO:4.

10. The method according to claim 7, wherein said beta cells are human pancreatic beta cells.

11. A method for treating diabetes in a subject, comprising: (a) obtaining beta cells from the subject being treated or a donor; (b) contacting the beta cells with an effective amount of: SDF-1 (the polypeptide having the amino acid sequence set forth in SEQ ID NO:1), a polypeptide having amino acid sequence substantially homologous thereto, or a fragment thereof capable of increasing beta cell survival; and GLP-1 (the polypeptide having the amino acid sequence set forth in SEQ ID NO:2), Exendin-4 (the polypeptide having the amino acid sequence set forth in SEQ ID NO:3), a polypeptide having amino acid sequence substantially homologous to GLP-1 or Exendin-1, or a fragment of GLP-1 or Exendin-1 capable of promoting beta cell proliferation; and (c) administering the beta cells that were treated in step (b) to the subject.

12. The method according to claim 11, wherein the fragment of GLP-1 capable of promoting beta cell proliferation with which the beta cells are contacted has the amino acid sequence set forth in SEQ ID NO:4.

13. The method according to claim 12, wherein the fragment of GLP-1 additionally comprises an amide moiety at the C-terminus of SEQ ID NO:4.

14. The method according to claim 11, wherein said subject is a human with Type 1 diabetes.

15. The method according to claim 11, wherein said subject is a human with Type 2 diabetes.

16. The method according to claim 11, wherein the beta cells contacted in step (b) are allowed to increase in mass before administration to the subject.

17. Use of SDF-1 (the polypeptide having the amino acid sequence set forth in SEQ ID NO:1), a polypeptide having amino acid sequence substantially homologous thereto, or a fragment thereof capable of increasing beta cell survival; and GLP-1 (the polypeptide having the amino acid sequence set forth in SEQ ID NO:2), Exendin-4 (the polypeptide having the amino acid sequence set forth in SEQ ID NO:3), a polypeptide having amino acid sequence substantially homologous to GLP-1 or Exendin-4, or a fragment of GLP-1 or Exendin-4 capable of promoting beta cell proliferation for the manufacture of a medicament for treating diabetes in a subject.

18. The use according to claim 17, wherein the fragment of GLP-1 capable of promoting beta cell proliferation has the amino acid sequence set forth in SEQ ID NO:4.

19. SDF-1 (the polypeptide having the amino acid sequence set forth in SEQ ID NO:1), a polypeptide having amino acid sequence substantially homologous thereto, or a fragment thereof capable of increasing beta cell survival; and GLP-1 (the polypeptide having the amino acid sequence set forth in SEQ ID NO:2), Exendin-4 (the polypeptide having the amino acid sequence set forth in SEQ ID NO:3), a polypeptide having amino acid sequence substantially homologous to GLP-1 or Exendin-4, or a fragment of GLP-1 or Exendin-4 capable of promoting beta cell proliferation for use in the treatment of diabetes in a subject.

20. The composition of claim 19 for use in the treatment of diabetes in a subject, comprising the fragment of GLP-1 capable of promoting beta cell proliferation, wherein said fragment has the amino acid sequence set forth in SEQ ID NO:4.
